(19) United States
(12) Patent Application Publication
(10) Pub. No.: US 2024/0131499 A1
(43) Pub. Date: Apr. 25, 2024

(54) BIOMIMETIC CATALYSTS FOR DIVERSE INDUSTRIAL APPLICATIONS IN CHEMICAL REACTIONS

(71) Applicant: Wisconsin Alumni Research Foundation, Madison, WI (US)

(72) Inventors: Hochan Chang, Madison, WI (US); George Huber, Middleton, WI (US); James Dumesic, Verona, WI (US)

(21) Appl. No.: 18/471,452

(22) Filed: Sep. 21, 2023

Related U.S. Application Data
(60) Provisional application No. 63/408,513, filed on Sep. 21, 2022.

(51) Int. Cl. B01J 31/04; C07D 491/18

(52) U.S. Cl. CPC B01J 31/04; C07D 491/18

(57) ABSTRACT

Organocatalysts can be engineered to mimic biological enzymes for use in a variety of industrially relevant chemical reactions. The organocatalyst comprises a chemical catalyst that is made by imidation of an amine-containing compound to form an N-substituted maleimide and appending the N-substituted maleimide on a furan-containing compound by Diels-Alder reaction and hydrogenation. The N-substituted maleimide may also be made by reacting a protected maleimide and compound containing a primary alkyl halide moiety. The organocatalysts are engineered to append chemical functionalities of amino acids such as glutamic acid for catalyzing hydrolysis of lactose. The organocatalyst further comprises a support to immobilize the chemical catalyst which plays the role of a scaffold to stabilize catalytic active sites and bind the reactants nearby the active sites, similar to a protein scaffold in biological catalysts.

### CROSS-REFERENCE TO RELATED APPLICATIONS

Priority is hereby claimed to provisional application Ser. No. 63/408,513, filed Sep. 21, 2022, which is incorporated herein by reference.

### FEDERAL FUNDING STATEMENT

This invention was made with government support under DE-SC0014058 awarded by the US Department of Energy. The government has certain rights in the invention.

### BACKGROUND

The cellulose fraction of lignocellulosic biomass is a sustainable carbon resource due to its natural abundance. Alonso, Wettstein, Dumesic, Bimetallic catalysts for upgrading of biomass to fuels and chemicals. Chem. Soc. Rev. 41, 8075-8098 (2012). 5-Hydroxymethyl furfural (HMF) has been developed as a renewable platform chemical for the synthesis of commodity chemicals, including liquid fuels and monomers for the production of polymers. Motagamwala, Won, Sener, Alonso, Maravelias, Dumesic, Toward biomass-derived renewable plastics: Production of 2,5-furandicarboxylic acid from fructose. Sci. Adv. 4 (2018). For example, HMF can be oxidized over metal catalysts and converted to 2,5-furan dicarboxylic acid (FDCA) for the synthesis of polyethylene furanoate (PEF) as a replacement for polyethylene terephthalate (PET). Davis, Houk, Tamargo, Datye, Davis, Oxidation of 5-hydroxymethylfurfural over supported Pt, Pd and Au catalysts. Catal. Today. 160, 55-60 (2011); De Jong, Dam, Sipos, Gruter, Furandicarboxylic acid (FDCA), A versatile building block for a very interesting class of polyesters. ACS Symp. Ser. 1105, 1-13 (2012). Hydrodeoxygenation of HMF can produce 2,5-dimethylfuran (DMF) which can be used directly as a gasoline additive or further upgraded to p-xylene after sequential Diels-Alder reaction and dehydration in the presence of zeolite catalysts. Williams, Chang, Do, Nikbin, Caratzoulas, Vlachos, R. F. Lobo, Fan, Dauenhauer, Cycloaddition of Biomass-Derived Furans for Catalytic Production of Renewable p-Xylene. ACS Catal. 2, 935-939 (2012). HMF can also be used to synthesize new platform chemicals that have higher molecular weight. For example, a HMF-derived platform chemical, named HAH, with higher molecular weight (274 g/mol) has been synthesized by aldol-condensation of HMF with acetone. Chang, Motagamwala, Huber, Dumesic, Synthesis of biomass-derived feedstocks for the polymers and fuels industries from 5-(hydroxymethyl)furfural (HMF) and acetone. Green Chem. 21, 5532-5540 (2019). The enone functionality of HAH absorbs ultraviolet (UV) light (378 nm), and HAH could potentially be used as a yellow organic dye. Chang, Bajaj, Huber, Maravelias, Dumesic, Catalytic strategy for conversion of fructose to organic dyes, polymers, and liquid fuels. Green Chem. 22, 5285-5295 (2020). Moreover, the diol group in HAH can be incorporated into polymer structures by forming polyurethanes and polyesters. Chang, Gilcher, Huber, Dumesic, Synthesis of performance-advantaged polyurethanes and polyesters from biomass-derived monomers by aldol-condensation of 5-hydroxymethyl furfural and hydrogenation. Green Chem. 23, 4355-4364 (2021). The furan moiety in HAH can be activated for Diels-Alder reaction by selective hydrogenation of the C═C bond in the enone group in HAH. Therefore, partially hydrogenated HAH, denoted as PHAH, can be used as a monomer for the synthesis of a property-tunable polymer by Diels-Alder reaction. Diels-Alder reactions are important reactions for biological applications because these reactions can append additional functionalities into the molecules, and the Diels-Alder bond is reversible at high temperature or in the presence of Brønsted acids. Gregoritza, Brandl, The Diels-Alder reaction: A powerful tool for the design of drug delivery systems and biomaterials. Eur. J. Pharm. Biopharm. 97, 438-453 (2015); Kotha, Banerjee, Recent developments in the retro-Diels-Alder reaction. RSC Adv. 3, 7642-7666 (2013).

Enzymes are used in dairy and biorefinery applications because of their superior activity and selectivity for desired chemical reactions. However, enzymes cannot be simply recollected after the reaction, and immobilization of enzymes can cause a decrease in activity. Moreover, enzymatic reactions require the use of biological media, containing nutrients in aqueous buffers, to achieve high activity and selectivity, and the use of buffer media gives rise to additional separation steps for downstream processes. These limitations of enzymatic reactions increase the production cost of the final products and can limit the selection of reaction conditions in downstream processes. Accordingly, development of chemical catalysts from sustainable resources that can function through mechanisms that are analogous to enzymatic processes can be of critical importance for sustainable and economical processes. Previous research on the synthesis of artificial enzymes has been limited by limited degrees of freedom to engineer the active sites on the supports. For example, the catalytic activities of silica and alumina for polyglucan hydrolysis were limited by the maximum density of the Brønsted acidic —OH sites. Gazit, Katz, Understanding the Role of Defect Sites in Glucan Hydrolysis on Surfaces. J. Am. Chem. Soc. 135, 4395-4402 (2013). Furthermore, the catalytic active sites are limited to the molecules with simple structures when the chemicals are added to supports by grafting methods. Chen, Brunelli, Investigating the Impact of Microporosity of Aminosilica Catalysts in Aldol Condensation Reactions for Biomass Upgrading of 5-Hydroxymethylfurfural and Furfuraldehyde to Fuels. Energy and Fuels. 35, 14885-14893 (2021). Thus, to address the long-felt need for sustainable artificial enzymes with increased activities, this disclosure presents new classes of biomimetic catalysts created by engineering active sites using biomass-derived molecules.

### SUMMARY

The engineered structures and active sites of enzyme catalysts give rise to high catalytic activity and selectivity toward desired reactions. Disclosed herein is an organocatalyst that mimics the chemical functional groups that comprise the active site channels in enzyme catalysts. The organocatalyst comprises a chemical catalyst having the following structure:

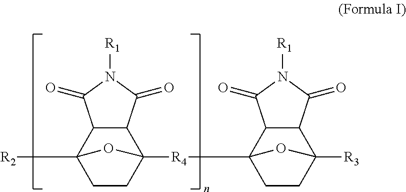

The R₁ substituent are chemical functionalities that catalyze chemical reactions. Each R₁ is independent of every other R₁. Thus, when “n≥2,” the multiple R₁'s in the formula can be the same or different chemical functionalities. In certain versions, R₁ is a moiety that catalyzes chemical reactions. In certain versions, R₁ is a moiety connected to the amine group of an amino acid (e.g., glutamic acid, tyrosine, taurine, and alanine).

The side chains R₂ and R₃ are each independently selected from the group consisting of hydrogen, hydroxy, halo, carboxyl, amine, alkyl, alkoxy, acyl, aryl, heteroalkyl, heteroalkoxy, heteroacyl, heteroaryl, and any combination of the foregoing. The linker R₄ can be absent (in which case the furan groups are directly connected), or selected from the group consisting of carboxyl, amino, alkylene, alkoxy, acyl, and any combination of the foregoing. “n” is an integer equal to or greater than 0. In certain versions, R₂ and R₃ are each hydroxymethyl, and R₄ is a C₁-C₂ₙ₊₁-di(alkyl) ketone i.e., —(CH₂)ₙ—CO—(CH₂)ₙ—, where “n” is an integer of from 1 to 6.

In a preferred version of the catalysts, when R₁ is the moiety connected to the amine group of glutamic acid, R₂ and R₃ are each hydroxymethyl, R₄ is —(CH₂)₂—CO—(CH₂)₂—, and n=1, which yields an exemplary organocatalyst having the following structure:

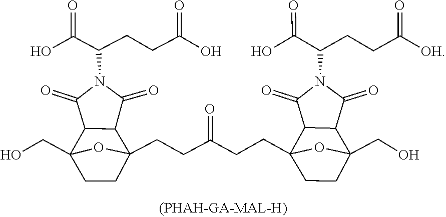

In some embodiments, the organocatalyst further comprises a support to immobilize the chemical catalyst. The support plays the role of a scaffold to stabilize catalytic active sites and bind the reactants nearby the active sites, similar to a protein scaffold in biological catalysts (enzymes).

Also disclosed herein is a method of making an organocatalyst that can be engineered to mimic biological enzymes for use in a variety of industrially relevant chemical reactions. The method comprises:
i) appending an amine-containing compound on an imido ring of maleic anhydride by imide formation (imidation) to form a N-substituted maleimide; and
ii) appending the N-substituted maleimide on a furan-containing compound by Diels-Alder reaction and hydrogenation.

The amine-containing compound comprises chemical functionalities that can catalyze chemical reactions. In certain versions, the amine-containing compound is an amino acid (e.g., glutamic acid, tyrosine, taurine, and alanine).

In certain versions, the furan-containing compound is a simple furan having one furan ring or a saturated analog thereof. In certain versions, the furan-containing compound is a compound having two or more furan rings or a saturated analog thereof. In some embodiments, the furan-containing compound having two or more furan rings is synthesized from aldol condensation of a simple furan. For example, the furan-containing compound is synthesized from aldol condensation and hydrogenation of 5-hydroxymethyl furfural (HMF) with acetone, and the terminal —OH group of the aldol condensation product can be oxidized to produce an aldehyde moiety for further aldol condensation with acetone and HMF to form complexes containing three or more furan groups. In some other embodiments, the furan-containing compound is synthesized by sequential aldol condensation of 2,5-diformylfuran (DFF) with acetone to form complexes containing three or more furan groups.

The method of making an organocatalyst may further comprise immobilizing the product of step (ii) on a support which plays the role of a scaffold to stabilize catalytic active sites and bind the reactants nearby the active sites, similar to a protein scaffold in biological catalysts (enzymes).

Also disclosed herein is an organocatalyst that catalyzes the hydrolysis of lactose. The organocatalyst comprises a chemical catalyst having the following structure:

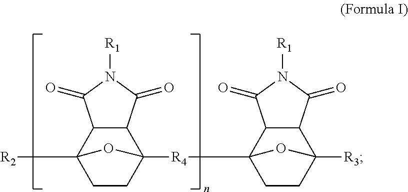

wherein each R₁ is a moiety connected to the amine functional group of amino acids or amines, and the amino acid and amine are (independently for each R₁) selected from the group consisting of glutamic acid, aspartic acid, tyrosine, taurine, glutamine, and alanine.

R₂ and R₃ are each independently selected from the group consisting of hydrogen, hydroxy, halo, carboxyl, amine, alkyl, alkoxy, acyl, aryl, heteroalkyl, heteroalkoxy, heteroacyl, heteroaryl, and any combination of the foregoing. R₄ can be absent (the furan groups are directly connected), or selected from the group consisting of carboxyl, amine, alkylene, alkoxy, acyl, and any combination of the foregoing functional groups. “n” is an integer equal to or greater than 0.

In preferred versions, R₁ is the moiety connected to the amine group of glutamic acid, R₂ and R₃ are each hydroxymethyl, and R₄ is —(CH₂)₂—CO—(CH₂)₂—. When n=1, a preferred exemplary chemical catalyst has the following structure:

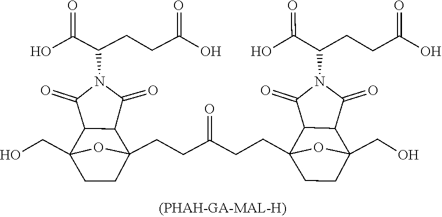

In preferred versions, the organocatalyst that catalyzes hydrolysis of lactose further comprises a support to immobilize the chemical catalyst. The support plays the role of scaffold to stabilize catalytic active sites and bind the reactants nearby, similar to a protein scaffold in biological catalysts (enzymes). In preferred embodiments, the support has an intermediate hydrophobicity. The support may be a functionalized silica support, a carbon-coating silica support, a N-doping support, an acidic support, or a carbon-based support. In some embodiments, the support is a functionalized silica support.

Also disclosed herein is a method of making an organocatalyst that catalyzes hydrolysis of lactose. The method comprises:
i) appending an amino acid or an amine selected from the group consisting of glutamic acid, aspartic acid, tyrosine, taurine, glutamine, and alanine on an imido ring of maleic anhydride by imidation to form a N-substituted maleimide; and
ii) appending the N-substituted maleimide on a furan-containing compound by Diels-Alder reaction and hydrogenation.

In certain versions, the furan-containing compound is a simple furan having one furan ring or a saturated analog thereof. In certain versions, the furan-containing compound is a compound having two or more furan rings or a saturated analog thereof. In some embodiments, the furan-containing compound having two or more furan rings is synthesized from aldol condensation of a simple furan. For example, the furan-containing compound is synthesized from aldol condensation and hydrogenation of 5-hydroxymethyl furfural (HMF) with acetone, and the terminal —OH group of the aldol condensation product can be oxidized to produce an aldehyde moiety for further aldol condensation with acetone and HMF to form complexes containing three or more furan groups. In some other embodiments, the furan-containing compound is synthesized by sequential aldol condensation of 2,5-diformylfuran (DFF) with acetone to form complexes containing three or more furan groups.

Preferably, the method of making an organocatalyst that catalyze hydrolysis of lactose further comprises immobilizing the product of step (ii) on a support which plays the role of scaffold to stabilize catalytic active sites and bind the reactants nearby, similar to a protein scaffold in biological catalysts (enzymes). In preferred embodiments, the support has an intermediate hydrophobicity. The support may be a functionalized silica support, a carbon-coating silica support, a N-doping support, an acidic support, or a carbon-based support. In some embodiments, the support is a functionalized silica support.

Also disclosed herein is a method of catalyzing hydrolysis of lactose. The method comprises contacting lactose with any of the organocatalysts that catalyze hydrolysis of lactose as disclosed herein, for a time and at a temperature to hydrolyze at least a portion of lactose to galactose.

In certain versions, the organocatalyst used in the method comprises a chemical catalyst having the following structure:

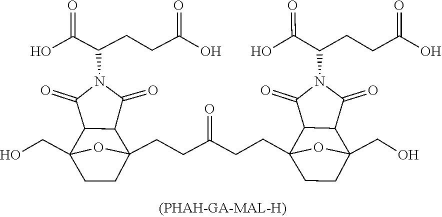

In preferred versions, the organocatalyst used in the method comprises a support to immobilize the chemical catalyst which plays the role of scaffold to stabilize catalytic active sites and bind the reactants nearby, similar to a protein scaffold in biological catalysts (enzymes). In preferred embodiments, the support has an intermediate hydrophobicity. The support may be a functionalized silica support, a carbon-coating silica support, a N-doping support, an acidic support, or a carbon-based support. In some embodiments, the support is a functionalized silica support.

The objects and advantages of the disclosure will appear more fully from the following detailed description of the preferred embodiment of the disclosure made in conjunction with the accompanying drawings.

### BRIEF DESCRIPTION OF THE DRAWINGS

FIG. 1A. Reaction pathways for the synthesis of organocatalysts and immobilization of PHAH-GA-MAL-H on various supports (colored molecules represent organocatalysts).

FIG. 1B. Graphical structure of the biomimetic organocatalyst (R— represents surface functionality of the support).

FIG. 1C. Parity plot of active site analysis by model fitting for the predicted and experimental rates for lactose hydrolysis.

FIG. 1D. Turnover frequency (TOF) for lactose hydrolysis as a function of the loading amounts of PHAH-GA-MAL-H on Si—C₄ support.

FIGS. 2A-2D. Effect of various supports on (FIG. 2A) catalytic activity (where rate constants were measured from the 1st cycle of hydrolysis), (FIG. 2B) catalyst stability, (FIG. 2C) catalyst selectivity, and (FIG. 2D) total turnover numbers (16 wt % loading of PHAH-GA-MAL-H on supports) for lactose hydrolysis. Reaction conditions: 420-435 mM of aqueous lactose solution was hydrolyzed at 100° C. in the presence of various catalysts; Rates for each hydrolysis cycle were measured by reaction kinetics analysis for 110-120 h of hydrolysis.

FIGS. 3A-3D. Reaction kinetics analysis for lactose hydrolysis (at 100° C.) in presence of different amino acids, FIG. 3A: taurine (kₐₚₚ=0.0011±0.0002 h⁻¹, k_{d,app}=0.0030±0.0056 h⁻¹). FIG. 3B: tyrosine (kₐₚₚ=0.0004±0.0001 h⁻¹, k_{d,app}=0.0005±0.0116 h⁻¹). FIG. 3C: alanine (kₐₚₚ=0.0005±0.0001 h⁻¹, k_{d,app}=0.0102±0.0106 h⁻¹). FIG. 3D: glutamic acid (kₐₚₚ=0.0060±0.0004 h⁻¹, k_{d,app}=0.0027±0.0018 h⁻¹). kₐₚₚ and k_{d,app} represent the apparent rate constants for lactose hydrolysis and glucose degradation, respectively and the values are evaluated within 95% confidential intervals.

FIGS. 4A and 4B. Reaction kinetics analysis for lactose hydrolysis (at 100° C.) in presence of (FIG. 4A) sulfuric acid (kₐₚₚ=0.1615±0.0075 h⁻¹, k_{d,app}=0.0039±0.0195 h⁻¹) and (FIG. 4B) acetic acid (kₐₚₚ=0.0015±0.0001 h⁻¹, k_{d,app}=0.0003±0.0015 h⁻¹) for the calculation of rate constants for H⁺ (k_{H}_{⁺}) and non-dissociated acetic acid (k_{AA}). kₐₚₚ and k_{d,app} represent the apparent rate constants for lactose hydrolysis and glucose degradation, respectively and the values are evaluated within 95% confidential intervals; Rate=kₐₚₚ·[Lactose]₀=k_{H+}·[H⁺]·[Lactose]₀ for sulfuric acid and Rate=kₐₚₚ·[Lactose]₀=(k_{H+}·[H⁺]+k_{AA}·[AA])·[Lactose]₀ (AA represents acetic acid).

FIG. 5. Theoretical derivation of Ca(OAc)₂ effect on [H⁺] and the normalized [H⁺] profile at 25° C. by the theoretical calculation.

FIGS. 6A-6D. Reaction kinetics analysis for lactose hydrolysis (at 100° C.) in the presence of different amounts of Ca(OAc)₂; where FIG. 6A: [Ca(OAc)₂]=0 mM, Rate=0.0026 M·h⁻¹, kₐₚₚ=0.0060±0.0004 h⁻¹, k_{d,app}=0.0027±0.0018 h⁻¹. FIG. 6B: [Ca(OAc)₂]=5 mM, Rate=0.0016 M·h⁻¹, kₐₚₚ=0.0037±0.0002 h⁻¹, k_{d,app}=0.0020±0.0013 h⁻¹). FIG. 6C: [Ca(OAc)₂]=16 mM, Rate=0.0007 M·h⁻¹, kₐₚₚ=0.0016±0.0001 h⁻¹, k_{d,app}=0.0023±0.0018 h⁻¹). FIG. 6D [Ca(OAc)₂]24 mM, Rate=0.0005 M·h⁻¹, kₐₚₚ=0.0011±0.0001 h⁻¹, k_{d,app}=0.0037±0.0017 h⁻¹). kₐₚₚ and k_{d,app} represent the apparent rate constants for lactose hydrolysis and glucose degradation, respectively and the values are evaluated within 95% confidential intervals. (Glutamic acid served as catalyst).

FIGS. 7A and 7B. Parity plots of active site analysis by model fitting for theoretical and experimental reaction rate. The active site was assumed as (FIG. 7A) a single active site of G₂H and (FIG. 7B) dual active sites of G₁H and G₂H (k_{H+}, k_{G2H}, and k_{G1H,G2H} represent the rate constant from H⁺, G₂H, and combination of G₁H and G₂H as an active site in glutamic acid moiety, respectively).

FIGS. 8A-8C. Reaction kinetics analysis for lactose hydrolysis over the homogeneous organocatalysts, (FIG. 8A) glutamic acid (kₐₚₚ=0.0060±0.0004 h⁻¹, k_{d,app}=0.0027±0.0018 h⁻¹), (FIG. 8B) GA-MAL-H (kₐₚₚ=0.0077±0.0002 h⁻¹, k_{d,app}=0.0022±0.0008 h⁻¹), and (FIG. 8C) PHAH-GA-MAL-H (kₐₚₚ=0.0117±0.0004 h⁻¹, k_{d,app}=0.0016±0.0005 h⁻¹). kₐₚₚ and k_{d,app} represent the apparent rate constants for lactose hydrolysis and glucose degradation, respectively and the values are evaluated within 95% confidential intervals.

FIG. 9. Conversion of GA-MAL by Diels-Alder reaction with PHAH over the reaction time (measured by HPLC).

FIGS. 10A and 10B. Reaction kinetics analysis for lactose hydrolysis (at 100° C.) in presence of (FIG. 10A) 37 mM (kₐₚₚ=0.0117±0.0004 h⁻¹, k_{d,app}=0.0016±0.0005 h⁻¹) and (FIG. 10B) 70 mM (kₐₚₚ=0.0103±0.0004 h⁻, k_{d,app}=0.0010±0.0006 h⁻¹) of homogeneous PHAH-GA-MAL-H. (kₐₚₚ: apparent rate constants for lactose hydrolysis, k_{d,app}: apparent rate constant for glucose degradation within 95% confidential intervals).

FIGS. 11A and 11B. Effect of catalyst loading amount on (FIG. 11A) apparent rate constant, and (FIG. 11B) catalyst stability (16 wt % PHAH-GA-MAL-H was supported on Si—C₄) for lactose hydrolysis.

FIGS. 12A-12E. Hydrothermal degradation during lactose hydrolysis at 100° C. of (FIG. 12A) glutamic acid, (FIG. 12B) GA-MAL-H, (FIG. 12C) homogeneous PHAH-GA-MAL-H, (FIG. 12D) PHAH-GA-MAL-H (43 wt % loading) on Si—C₄, and (FIG. 12E) PHAH-GA-MAL-H (43 wt % loading) on Si-diol. (Degradation of PHAH-GA-MAL-H produced glutamic acids and the yield of glutamic acid was analyzed by HPLC; [PHAH-GA-MAL-H]=[PHAH-GA-MAL-H]₀—[Glutamic acid]/2).

FIGS. 13A-13D. Catalyst activity (turnover frequency, TOF) over reaction time at different reaction temperature (FIG. 13A) 120° C., (FIG. 13BA) 100° C., (FIG. 13C) 90° C. and (FIG. 13D) Van't hoff plot for catalyst deactivation (blue) and lactose hydrolysis (red); Activation energy for catalyst deactivation and lactose hydrolysis is 116.2 and 106.7 kJ/mol, respectively.

FIG. 14. Catalyst stability analysis for the non-deactivating immobilized catalyst (16 wt % loading of PHAH-GA-MAL-H was supported on silicon-carbide); Slope represents rate constant for catalyst deactivation (k_{deact.}˜0 h⁻¹) and the averaged rates were used as the rate for lactose hydrolysis (Rate=0.0010 M·h⁻¹) and used to calculate k_{G}_{₁}_{H} and TOF for the active site

FIGS. 15A-15L. Catalyst stability analysis for the deactivating immobilized catalyst (16 wt % loading of PHAH-GA-MAL-H) on various supports, (FIG. 15A) Si-diol, (FIG. 15B) Si—C₁, (FIG. 15C) Si—C₄, (FIG. 15D) Si—C₁₈, (FIG. 15E) C-coated Si (by pyrolysis at 200° C.), (FIG. 15F) C-coated Si (by pyrolysis at 300° C.), (FIG. 15G) C-coated Si (by pyrolysis at 400° C.), (FIG. 15H) C-coated SBA-15 with 4 nm pores (by pyrolysis at 400° C.), (FIG. 15I) C-coated SBA-15 with 6 nm pores (by pyrolysis at 400° C.), (FIG. 15J) Acetamide-coated Si (by pyrolysis at 200° C.), (FIG. 15K) Acetamide-coated Si (by pyrolysis at 400° C.), (FIG. 15L) TiO₂; Slope represents the rate constant for catalyst deactivation (k_{deact.}), y-intercept shows the log-scale initial rate for lactose hydrolysis (ln(Rate₀) and Rate₀ was used to calculate k_{G}_{₁}_{H} and TOF for the active site.

FIGS. 16A-16D. Reaction kinetics analysis for lactose hydrolysis (at 100° C.) over the (FIG. 16A) TiO₂ (kₐₚₚ=0.0021±0.0001 h⁻¹, k_{d,app}=0.0064±0.0023 h⁻¹), (FIG. 16B) Si-diol (kₐₚₚ=0.0005±0.0001 h⁻¹, k_{d,app}=−0.0031±0.0029 h⁻¹), (FIG. 16C) Si—C₄ (kₐₚₚ=0.0007±0.0001 h⁻¹, k_{d,app}=0.0010±0.0035 h⁻¹) (0.16 g of bare support), and (FIG. 16D) PHAH-GA-MAL-H (16 wt % loading) on TiO₂ (kₐₚₚ=0.0065±0.0002 h⁻¹, k_{d,app}=0.0040±0.0007 h⁻¹); (kₐₚₚ: apparent rate constants for lactose hydrolysis, k_{d,app}: apparent rate constant for glucose degradation within 95% confidential intervals).

### DETAILED DESCRIPTION

### Abbreviations and Definitions

HPLC=High-performance liquid chromatography; qNMR=Quantitative Nuclear Magnetic Resonance; 2D HSQC NMR=2-Dimensional Heteronuclear Single Quantum Coherence Nuclear Magnetic Resonance; GC-FID=Gas Chromatography Flame Ionization Detection; DA adduct=Diels-Alder adduct.

As used herein, the terms “furan” or “simple furan” refer to a class of organic compounds containing the traditional furan ring structure as well as a saturated analog thereof. A furan ring is considered electron rich because it has higher electron/proton ratio than benzene. Examples of furan include, but not limited to furan, furfural, furfuryl alcohol, 5-hydroxymethyl-2-furancarboxyaldehyde, 5-methyl-2-furancarboxyaldehyde, 2-vinyl furoate, 5-methyl-2-vinylfuroate; 5-tertbutyl-2-vinyl furoate, 2-furfurylmethacrylate, 2-furfuryl methylmethacrylate, 2-vinyl furan, 5-methyl-2-vinyl furan, 2-(2-propylene)furan (or 2-methyl vinylidene furan), 5-methyl-2-methyl vinylidenefuran; furfurylidene acetone, 5-methyl-2-furfurylidene acetone, 2-vinyl tetrahydrofuran, 2-furyl oxirane, 5-methyl-2-furyloxirane, furfuryl vinyl ether, 5-methyl-furfuryl vinyl ether, vinyl 2-furyl ketone, bis-2,5-carboxyaldehyde furan, bis-2,5-hydroxymethyl furan, 5-hydroxymethyl-2-ethyl furanacrylate, 2,5-furandicarboxylic acid, 2,5-furan diacid dichloride, 2,5-furan dicarboxylic acid dimethyl ester, 2,5-furan methylamine, 5-carboxy-2-furan amine, 5-methylester-2-furan amine, bis-(2,5-methylene isocyanate) furan, bis(2,5-isocyante) furan, 2-isocyanate furyl, and 2-methylene isocyanate furyl.
HMF=5-hydroxymethyl furfural:

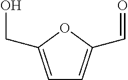

DFF=2,5-diformylfuran:

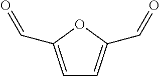

HA=5-(hydroxymethyl)-furan-2-butenone:

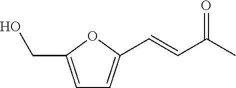

HAH=di-((5-hydroxymethyl)furan-2-ethenyl)ketone:

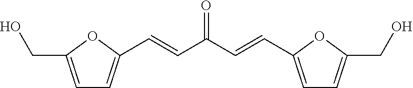 [SMILES: O=C(/C=C/c1ccc(CO)o1)/C=C/c1ccc(CO)o1]

PHAH=partially-hydrogenated HAH:

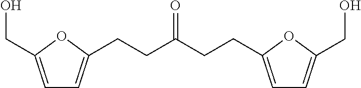 [SMILES: O=C(CCc1ccc(CO)o1)CCc1ccc(CO)o1]

“Maleimide” refers to compounds having the structure:

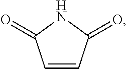

and N-substituted analogs thereof.
GA=Glutamic acid:

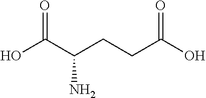

GA-MAL: imidation reaction product of GA and maleic anhydride:

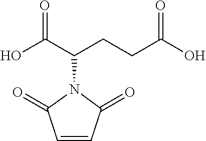

GA-MAL-H: hydrogenated GA-MAL:

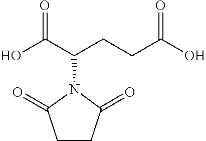

PHAH-GA-MAL: Diels-Alder adduct of GA-MAL and PHAH:

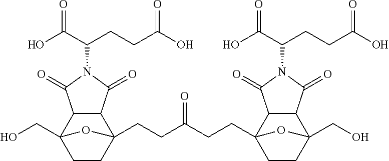

PHAH-GA-MAL-H: hydrogenated PHAH-GA-MAL:

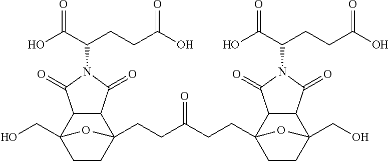

The imidation reaction is conversion of a compound (especially an amine) into an imide. In organic chemistry, an “imide” is a functional group consisting of two acyl groups bound to nitrogen.

The aldol reaction is a reaction in which an enol or an enolate ion reacts with a carbonyl compound to form a β-hydroxyaldehyde or a β-hydroxyketone. The β-hydroxyaldehyde or a β-hydroxyketone can then be dehydrated to give a conjugated enone. Colloquially, the first step is often called an “aldol reaction,” while the two-step reaction to yield the conjugated enone is referred to as an “aldol condensation.”

The Diels-Alder reaction is a reaction between a conjugated diene and a substituted alkene to yield a substituted cyclohexene derivative. The imidation, aldol condensation, and Diels-Alder reactions are well known to chemists of ordinary skill in the art and will not be described in any detail herein.

The Gabriel synthesis is a chemical reaction that transforms primary alkyl halides into amines. (See, for example, Gibson, M. S.; Bradshaw, R. W. (1968). “The Gabriel Synthesis of Primary Amines”. Angew. Chem. Int. Ed. Engl. 7 (12): 919; and Ragnarsson and Greh (1991). “Novel Gabriel Reagents”. Acc. Chem. Res. 24(10): 285-289.)

As used herein, the terms “support”, “catalyst support”, “engineered support”, “support material” or “supporting material” refer to materials, usually a solid with a high surface area and surface functionality, to which a catalyst is affixed. (Surface functionalities can include, for example, hydrophobic or hydrophilic functionality, acidic or basic functionalities, etc.) Typical supports include various types of carbon, alumina, and silica-based materials.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 1 to 9, from 3.6 to 4.6, from 3.5 to 9.9, and so forth.

All references to singular characteristics or limitations of the present disclosure shall include the corresponding plural characteristic or limitation, and vice-versa, unless otherwise specified or clearly implied to the contrary by the context in which the reference is made. That is, unless specifically stated to the contrary, “a” and “an” mean “one or more.” The phrase “one or more” is readily understood by one of skill in the art, particularly when read in context of its usage. For example, “one or more” substituents on a phenyl ring designates one to five substituents.

As used herein, the term “or” is an inclusive “or” operator and is equivalent to the term “and/or” unless the context clearly dictates otherwise.

The elements and method steps described herein can be used in any combination whether explicitly described or not, unless otherwise specified or clearly implied to the contrary by the context in which the referenced combination is made.

All combinations of method or process steps as used herein can be performed in any order, unless otherwise specified or clearly implied to the contrary by the context in which the referenced combination is made.

The methods of the present disclosure can comprise, consist of, or consist essentially of the essential elements and limitations of the method described herein, as well as any additional or optional ingredients, components, or limitations described herein or otherwise useful in synthetic organic chemistry.

It is understood that the disclosure is not confined to the particular elements and method steps herein illustrated and described, but embraces such modified forms thereof as come within the scope of the claims.

### Biomimetic Organocatalysts

Disclosed herein is an organocatalyst that mimics the chemical functional groups that comprise the active site channels in enzyme catalysts. The organocatalyst comprises a chemical catalyst having the following formula:

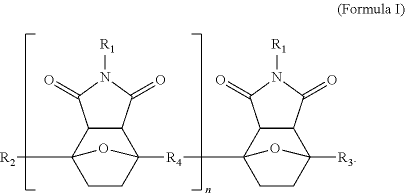

The organocatalysts can catalyze various chemical reactions when the N-group (R₁) and side chains (R₂ and R₃) are tailored appropriately, including the ability to append diverse chemical functionalities, such as amino acids.

Each independent R₁ in Formula I may be a chemical functionality that catalyzes chemical reactions. The multiple R₁ moieties can be the same or different chemical functionalities that catalyze chemical reactions.

Each R₁, independent of every other R₁, may be selected from the group consisting of hydrogen, halogen, optionally substituted alkyl, optionally substituted alkenyl, optionally substituted alkynyl, optionally substituted cycloalkyl, optionally substituted cycloalkenyl, hydroxyl, carboxyl, optionally substituted alkyloxy, optionally substituted alkenyloxy, optionally substituted alkynyloxy, optionally substituted cycloalkyloxy, optionally substituted cycloalkenyloxy, thiol, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, or an optionally substituted non-aromatic heterocyclic group.

Each optionally substituted alkyl, optionally substituted alkyloxy, optionally substituted alkylthio, optionally substituted alkylsulfinyl, optionally substituted alkylsulfonyl, optionally substituted alkylsulfonyloxy, and optionally substituted alkyloxycarbonyl, when substituted, may independently be substituted with one to three substituent(s) selected from the group consisting of cycloalkyl, alkylene optionally containing one or two heteroatom(s), hydroxyl, oxo, alkyloxy optionally substituted with a substituent group A at one to three position(s), thiol, alkylthio, halogen, nitro, cyano, carboxyl, alkyloxycarbonyl, optionally substituted amino, optionally substituted carbamoyl, acyl, aryl optionally substituted with a substituent group B at one to three position(s), heteroaryl optionally substituted with a substituent group C at one to three position(s), an optionally substituted non-aromatic heterocyclic ring group optionally substituted with a substituent group C at one to three position(s), aryloxy optionally substituted with a substituent group B at one to three position(s), and alkylsulfonyl;
each optionally substituted alkenyl, optionally substituted alkynyl, optionally substituted alkenyloxy, optionally substituted alkynyloxy, optionally substituted alkenylthio, optionally substituted alkynylthio, optionally substituted alkenyloxycarbonyl, optionally substituted alkynyloxycarbonyl, optionally substituted cycloalkyl, optionally substituted cycloalkenyl, optionally substituted cycloalkyloxy, optionally substituted cycloalkenyloxy, optionally substituted cycloalkylthio, optionally substituted cycloalkenylthio, optionally substituted cycloalkylsulfinyl, optionally substituted cycloalkenylsulfinyl, optionally substituted cycloalkylsulfonyl, optionally substituted cycloalkenylsulfonyl, optionally substituted cycloalkylsulfonyloxy, optionally substituted cycloalkenylsulfonyloxy, and optionally substituted alkylene optionally containing one or two heteroatom(s), when substituted, is independently substituted with one or more substituent(s) selected from the group consisting of alkyl optionally substituted with a substituent group D at one to three position(s), cycloalkyl, hydroxyl, oxo, alkyloxy optionally substituted with a substituent group A at one to three position(s), thiol, alkylthio, halogen, nitro, cyano, carboxyl, alkyloxycarbonyl, optionally substituted amino, optionally substituted carbamoyl, acyl, acyloxy, aryl optionally substituted with a substituent group B at one to three position(s), heteroaryl optionally substituted with a substituent group C at one to three position(s), non-aromatic heterocyclic group optionally substituted with a substituent group C at one to three position(s), aryloxy optionally substituted with a substituent group C at one to three position(s), and alkylsulfonyl;
each optionally substituted aryl, optionally substituted aryloxy, optionally substituted aryloxycarbonyl, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, and optionally substituted non-aromatic heterocyclic group, when substituted, are each independently substituted with one or more substituent(s) selected from the group consisting of alkyl optionally substituted with a substituent group D at one to three position(s), oxo, cycloalkyl, alkenyl, alkynyl, hydroxyl, alkyloxy optionally substituted with a substituent group A at one to three position(s), aryloxy optionally substituted with a substituent group B at one to three position(s), thiol, alkylthio, halogen, nitro, cyano, carboxyl, alkyloxycarbonyl, acyl, alkylsulfonyl, optionally substituted amino, optionally substituted carbamoyl, aryl optionally substituted with a substituent group B at one to three position(s), heteroaryl optionally substituted with a substituent group C at one to three position(s), and non-aromatic heterocyclic group optionally substituted with a substituent group C at one to three position(s);
each optionally substituted amino, optionally substituted carbamoyl, and optionally substituted sulfamoyl, when substituted, is independently substituted with one or two substituent(s) selected from the group consisting of alkyl, alkenyl, alkynyl, cycloalkyl, cycloalkynyl, aryl, heteroaryl, acyl, alkyloxycarbonyl, alkenyloxycarbonyl, alkynyloxycarbonyl, alkyl sulfonyl, alkenylsulfonyl, alkynylsulfonyl, arylsulfonyl, and heteroarylsulfonyl.
each substituent group A is independently selected from the group consisting of halogen and phenyl optionally substituted with one to three substituent(s) selected from substituent group B;
each substituent group B is independently selected from the group consisting of halogen, alkyl, alkyloxy, cyano, and nitro;
each substituent group C is independently selected from the group consisting of halogen and alkyl; and
each substituent group D is independently selected from the group consisting of halogen and alkyloxy.

The side chains R₂ and R₃ are each independently selected from the group consisting of hydrogen, hydroxy, halo, carboxyl, amine, alkyl, alkoxy, acyl, aryl, heteroalkyl, heteroalkoxy, heteroacyl, heteroaryl, and any combination of the foregoing. The linker R₄ can be absent (the furan groups are directly connected), or selected from the group consisting of halo, carboxyl, amine, alkyl, alkoxy, acyl, and any combination of the foregoing functional groups.

“n” is an integer equal to or greater than 0. For example, when n=0, the chemical catalyst has the following structure:

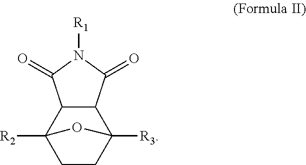

When n=1, the chemical catalyst has the following structure:

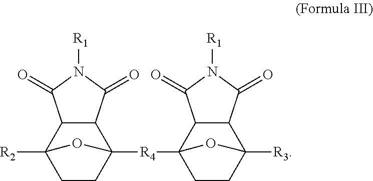

When n=2. the chemical catalyst has the following structure:

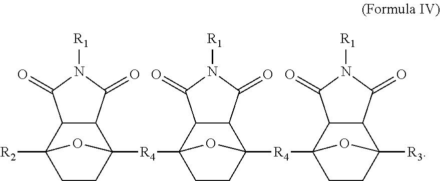

In certain versions, R₂ and R₃ are each hydroxymethyl, and R₄ is —(CH₂)₂—CO—(CH₂)₂—.

When R₂ and R₃ are each hydroxymethyl, R₄ is —(CH₂)₂—CO—(CH₂)₂—, and n=1 in Formula I, the chemical catalyst has following structure:

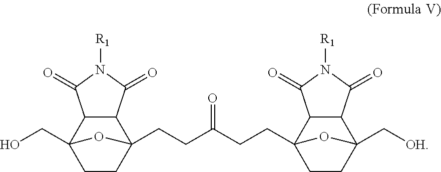

When R₁ in Formula V is the moiety connected to the amine group of glutamic acid, an exemplary chemical catalyst has the following structure:

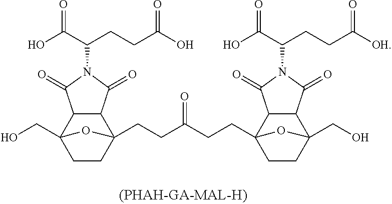

In some embodiments, the organocatalyst further comprises a support to immobilize the chemical catalyst. The support plays a role of a scaffold to stabilize catalytic active sites and bind the reactants nearby the active sites, similar to a protein scaffold in biological catalysts (enzymes). In general, materials for catalyst supports show high surface area, chemical stability as well as capability for dispersing the catalyst highly over the surface. The chemical and physical properties of surfaces affect to the activation of the supported catalysts to generate the characteristic properties of the catalysts. Well-defined structure and surface functionality of the support can tailor catalytic activity by affecting the chemical and physical interactions between the active sites and reactants on the surface of the support.

The support can be manufactured from a variety of materials. The manufactured support can have various pore sizes, structures, and chemical coatings. Examples of the support include, but are not limited to functionalized silica supports, carbon-coated silica supports, N-doping supports, acidic supports, and carbon-based support. Some specific examples are diol-functionalized silica, C1-functionalized silica, C4-functionalized silica, C18 functionalized silica, carbon-coated silica, amide-coated silica, inorganic oxides such as TiO₂, Al₂O₃, and inorganic carbides such as silicon-carbide. Methods of immobilizing catalysts on a support is known in the art, such as the methods described in Pham H. N. et al., ACS Catal. 2015, 5, 4546-4555 and Munnik P. et al., Chem. Rev. 2015, 115, 14, 6687-6718.

### Method of Synthesis

Disclosed herein is a method of synthesizing an organocatalyst that can be engineered to mimic biological enzymes for use in a variety of industrially relevant chemical reactions. The organocatalyst comprises a chemical catalyst having a structure of Formula I.

The first step of the method is to chemically append an amine-containing compound on the imido ring of maleic anhydride by imidation to form a N-substituted maleimide (Scheme 1).

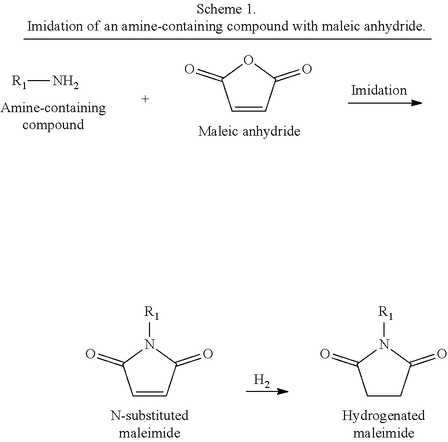

The second step of the method is to chemically append the N-substituted maleimides on a furan-containing compound, i.e., an electron-rich furan, by Diels-Alder reaction and hydrogenation (Schemes 2). Hydrogenation increases the stability against hydrothermal degradation of the organocatalyst and prevents side-reactions.

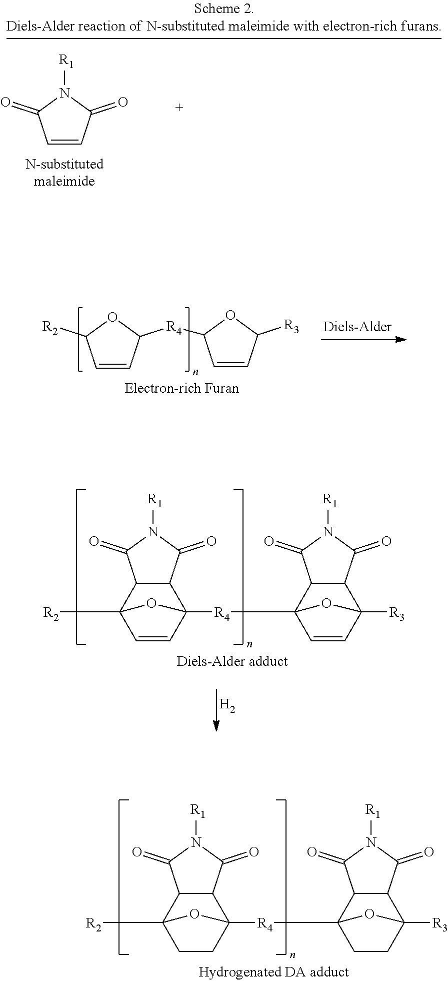

Optionally, the third step of the method is to immobilize the chemical catalyst on a supporting material (scheme 3) which plays a role of a scaffold to stabilize catalytic active sites and bind the reactants nearby the active sites, similar to a protein scaffold in biological catalysts (enzymes).

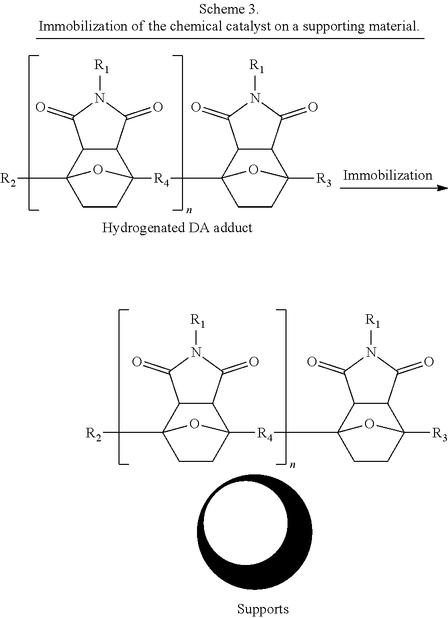

The amine-containing compound contemplated to be useful in the method includes any chemical comprising one or more amine groups, for example, primary amines, secondary amines, tertiary amines, diamines, polyamines, amino acids, amino sulfonic acida etc. In some versions, the amine-containing compounds are amino acids, including various natural and unnatural amino acids, of any chirality or mixture of chiralities. The amine-containing compound comprises chemical functionalities that can catalyze chemical reactions, such as active sites of amino acids.

The furan-containing compound contemplated to be useful in the method has the following structure:

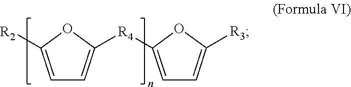

wherein R₂, R₃ and R₄ are as described above, and “n” is an integer equal to or greater than 0.

The furan-containing compound includes simple furans having one furan ring (when n=0), compounds having two or more furan rings (when n≥1), and the saturated analogs thereof.

The furan-containing compounds, i.e., the electron-rich furans can be made from biomass-derived furans. For example, a compound comprising two furan rings, named PHAH, can be produced by aldol-condensation and hydrogenation of a renewable biomass-derived platform chemical, 5-hydroxymethyl furfural (HMF). See Gilcher E. B. et al., Green Chem., 2022, 24, 2146-2159 and Gilcher E. B et al., ACS catalysis, 2022, 12, 10186-10198. Scheme 4 shows synthesis of PHAH from aldol condensation of HMF and acetone, followed by hydrogenation.

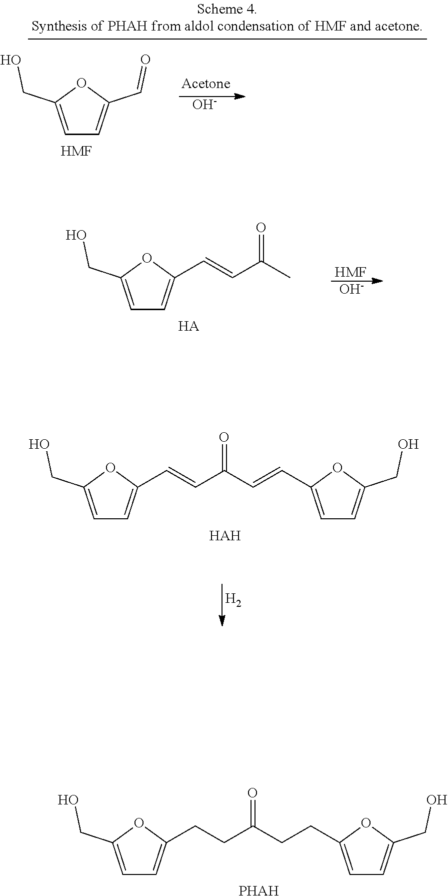

Larger complexes containing three or more furan rings can be formed by sequential aldol condensation reactions of diformylfuran (DFF) with acetone (Scheme 5), or oxidation of the terminal —OH groups of HAH to produce aldehyde moieties for aldol condensation with acetone and HMF (Scheme 6).

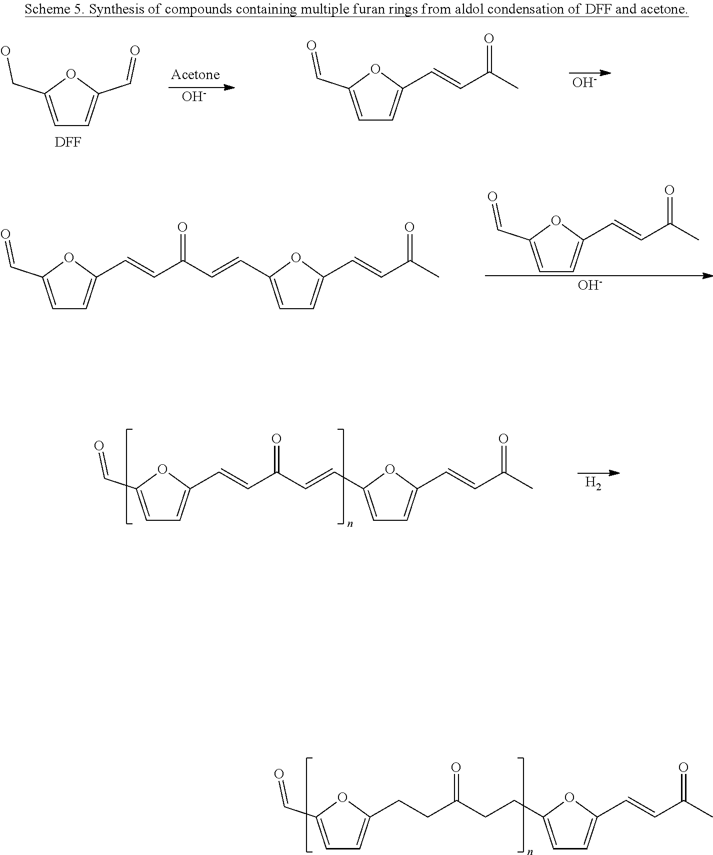

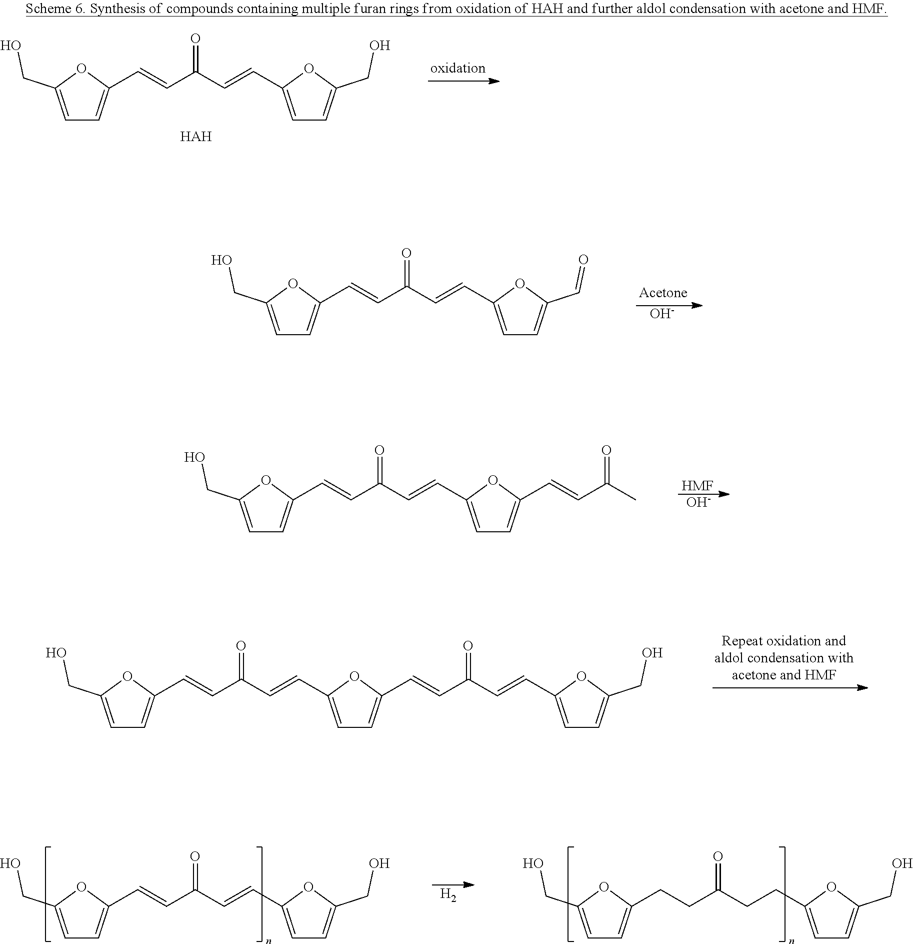

The electron rich furans provide active sites for functionalization by Diels-Alder reactions with N-substituted maleimides to append diverse chemical functionalities, such as amino acids. The Diels-Alder adducts can then catalyze various chemical reactions when the N-group and side chains of the DA adducts are tailored appropriately.

When multiple furan groups are present in the backbone of the material, it is possible to functionalize multiple chemical functionalities and thereby form constructs that are analogous to active sites in enzyme catalysts. More active catalysts formed in the manner of larger complexes containing three or more furan groups allow for operation at lower temperatures, closer to those temperatures used for enzymatic reactions.

Alternatively, the functionalized maleimide by reacting the N—H bond in maleimide (MAL-H) with a R¹—Cl bond to make MAL-R¹. The N—H bond in MAL-H is quite acidic and it reacts without catalyst with R—Cl bonds via the Gabriel synthesis. Coupled with the synthetic route disclosed above, this allows organocatalysts to be made in which the R¹ moiety is essentially unlimited. By way of example, compounds according to the present disclosure can be made in which R¹ is imidazole. Here, maleimide (MAL) and furan (FUR) are reacted via a Diels-Alder reaction to form a protected maleimide (FUR-MAL). See Scheme 7. In Scheme 7, the double bond in maleimide is protected by the fused furan ring.

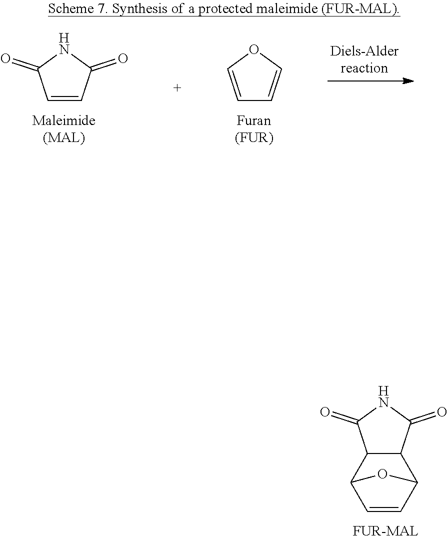 [SMILES: O=C1C=CC(=O)N1 ; c1ccoc1 ; O=C1NC(=O)C2C3C=CC(O3)C12]

The R¹ moiety, in this case an imidazole group, is attached to FUR-MAL by reacting the corresponding chloromethyl-imidazole (IZ-MeCl) with the FUR-MAL (Gabriel synthesis), as shown in Scheme 8.

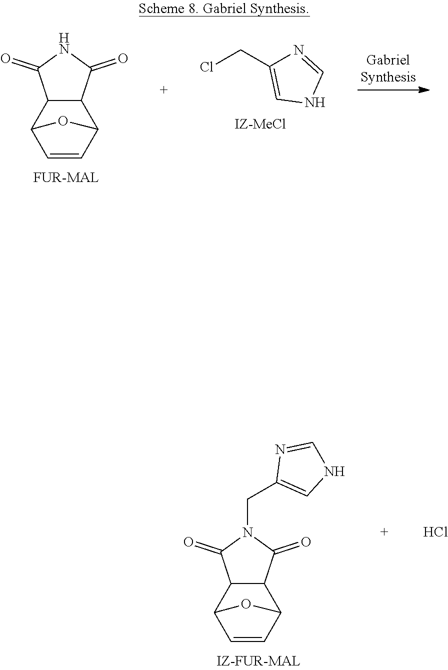 [SMILES: O=C1NC(=O)C2C3C=CC(O3)C12 ; ClCc1c[nH]cn1 ; O=C1C2C3C=CC(O3)C2C(=O)N1Cc1c[nH]cn1 ; Cl]

A retro Diels-Alder reaction is then used to remove the furan protecting group, thereby generating the imidazole-substituted maleimide (i.e., R¹=N-4-methyl imidazolyl), as shown in Scheme 9.

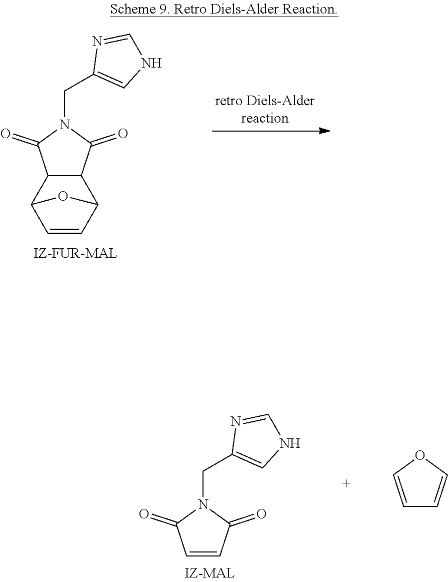 [SMILES: O=C1C2C3C=CC(O3)C2C(=O)N1Cc1c[nH]cn1 ; O=C1C=CC(=O)N1Cc1c[nH]cn1 ; c1ccoc1]

The IZ-MAL molecule is then reacted with, for example, PHAH via a Diels-Alder reaction. Subsequent selective hydrogenation, as described above, yields the final organocatalyst. See Scheme 10.

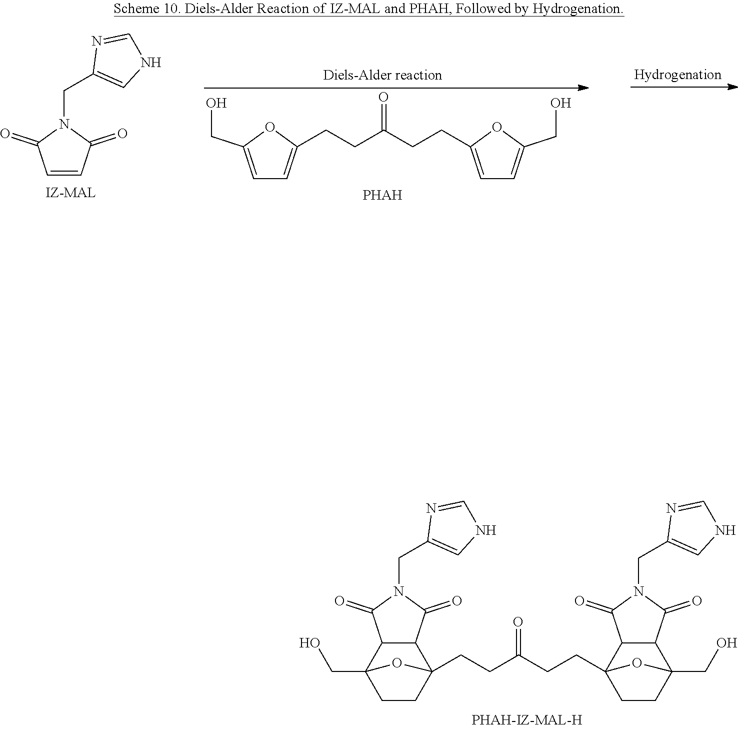 [SMILES: O=C1C=CC(=O)N1Cc1c[nH]cn1 ; O=C(CCc1ccc(CO)o1)CCc1ccc(CO)o1 ; O=C(CCC12CCC(CO)(O1)C1C(=O)N(Cc3c[nH]cn3)C(=O)C12)CCC12CCC(CO)(O1)C1C(=O)N(Cc3c[nH]cn3)C(=O)C12]

Thus, the overall reaction can be extended to any functional group (R₁, R₂, etc.) that can be attached to the nitrogen atom of the maleimide, as shown in Scheme 11:

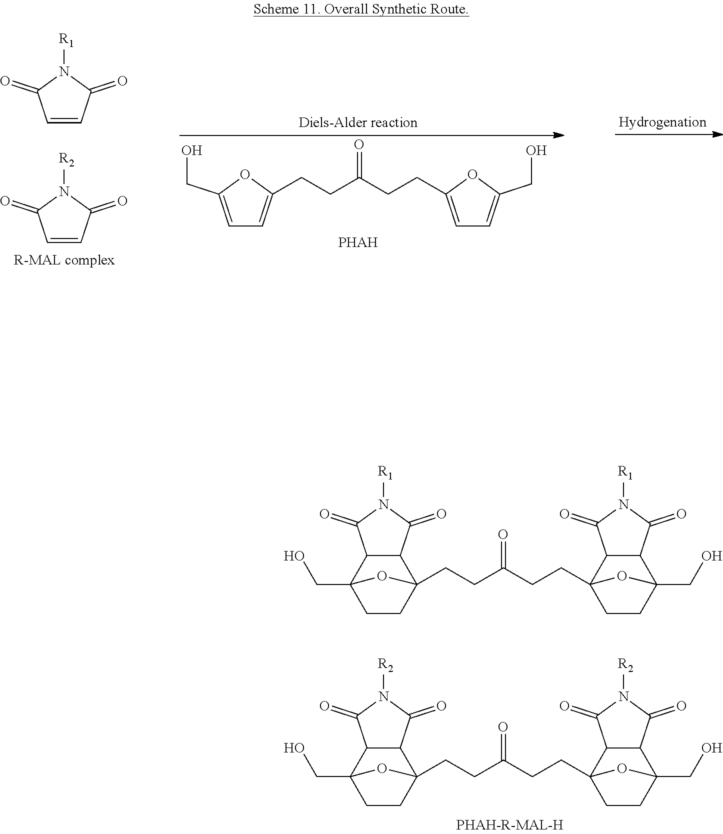

The resulting organocatalyst may then be adhered to a support by any means know known or developed in the future, such as by wetness impregnation.

The biomimetic organocatalysts disclosed herein are advantageous for efficient downstream processes because they do not require biological broths, containing nutrients in aqueous buffers, for operation, compared to enzymes. The further advantages of the biomimetic organocatalysts are that they can tailor the appropriate catalytic properties for various reactions by engineering the nature of the active sites and the structures and surface properties of the support to be analogous to enzyme catalysts.

The organocatalysts have high degrees of freedom for tailoring the chemical functionalities on the chemical catalyst and support materials. For example, imidation of maleic anhydride with diamines or tertiary amines can produce organocatalysts for base-catalyzed reactions, such as Aldol condensation. The organocatalysts may be synthesized from N-substituted maleimides with various amino acids (e.g., tyrosine, aspartic acid, glutamine) and immobilizing the organocatalysts with different ratios of amino acids to mimic the active site in enzymes for hydrolysis of polysaccharides.

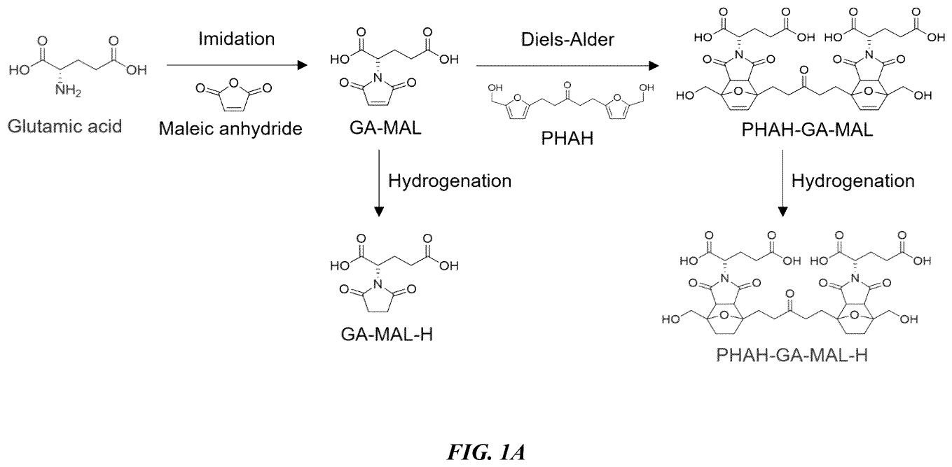

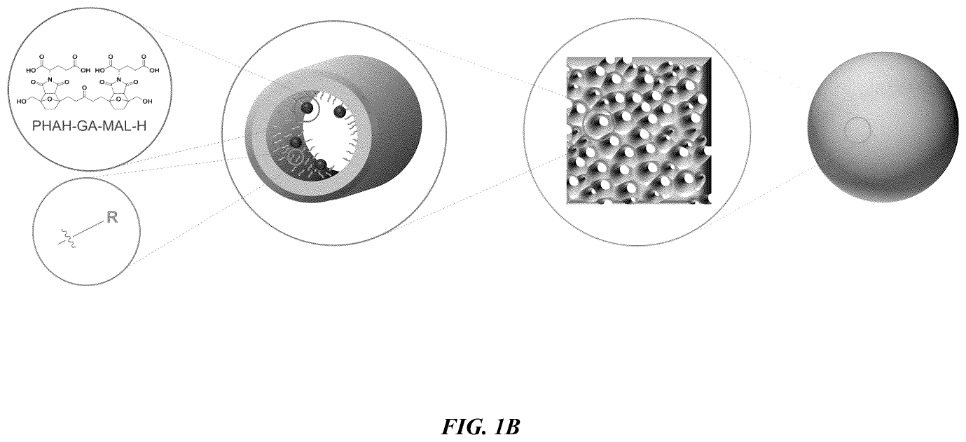

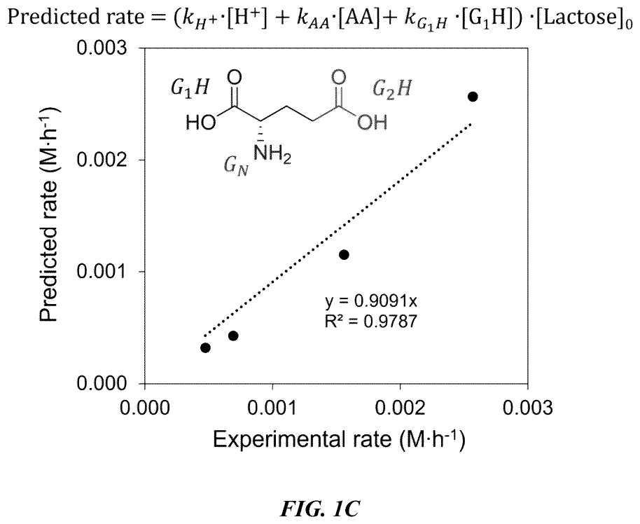

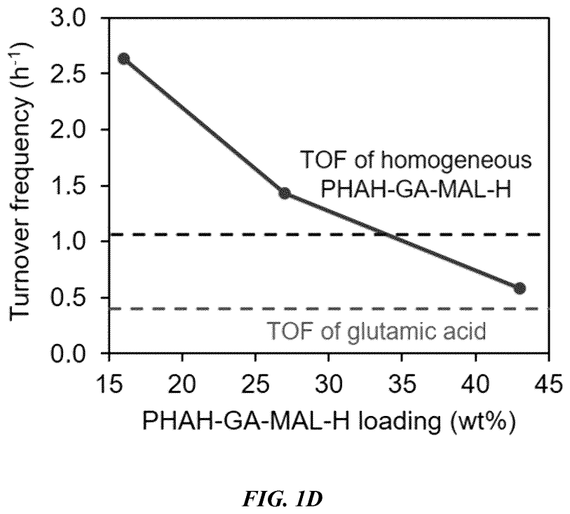

The following Example section describes an exemplary organocatalyst (PHAH-GA-MAL-H) that is made from biomass-derived difuran compounds and can be used for lactose hydrolysis. Lactose hydrolysis is an important reaction in the food and dairy industries. Also, lactose hydrolysis produces galactose as a precursor for synthesis of the low-calorie value-added sugar tagatose. The following Examples demonstrate the catalytic properties of the organocatalyst for lactose hydrolysis. The organocatalyst was engineered by imidation, Diels-Alder reaction, and hydrogenation (FIG. 1A). The organocatalyst complexes were then deposited onto well-defined solid supports resembling the protein framework in enzyme catalysts. A chemical-coating strategy by pyrolysis was developed to prepare supporting materials that have different surface functionality for catalyst immobilization and chemical reaction. The immobilized organocatalyst displayed catalytic activity for lactose hydrolysis 5.2 times higher than commercial glutamic acid which is a chemical functionality in the active sites of lactase.

It is highlighted that the organocatalysts disclosed herein are used for diverse chemical reactions by appending different chemical functionalities on furan active sites and using different surface properties of the supporting materials, not limiting to the examples provided herein.

### EXAMPLES

### Summary

This Example employs biomass-derived difuran compounds to append N-substituted maleimides with amino acid substitutions by Diels-Alder reaction to mimic the chemical functional groups that comprise the active site channels in enzyme catalysts. The biomass-derived Diels-Alder adducts, denoted as a norcantharimides, append two amino acid moieties to form an organocatalyst, and the catalytic activity of this moiety can be improved by immobilization on supports with well-defined pore architectures and surface properties. Accordingly, the structures of these immobilized organocatalysts can be engineered to mimic enzymatic active sites and to control the interaction between reactants, products, and transition states of catalytic reactions. It is demonstrated that the catalytic properties of such biomimetic catalysts for lactose hydrolysis to provide an example for an important industrial application.

### Experimental Methods

### Materials:

5-hydroxymethy furfural (HMF, AK Scientific (Union City, California), 98%), tetrahydrofuran (THF, Sigma-Aldrich (St. Louis, Missouri), 250 ppm BHT as inhibitor, ACS reagent ≥99.0%), acetone (Fisher Chemical (Fairlawn, New Jersey), HPLC grade), NaOH (Honeywell (Charlotte, North Carolina), Reagent grade, pellets (anhydrous), ≥98%), HCl (Sigma-Aldrich, 37%), Mill-Q water (MQ water, ˜18 MΩ cm), L-glutamic acid (Sigma-Aldrich, ReagentPlus, ≥99%), maleic anhydride (Fluka, puriss.: ≥99.0%), acetic acid (Sigma-Aldrich, ReagentPlus, ≥99%), sulfuric acid solution (Fluka (Charlotte, North Carolina), Reag. Ph. Eur., volumetric, 0.5M), β-alanine (Sigma-Aldrich, BioReagent), L-tyrosine (Sigma-Aldrich, BioUltra, ≥99.0%), taurine (Fluka, puriss. p,a,: >99.0%), calcium acetate hydrate (Ca(OAc)₂, Sigma-Aldrich, BioUltra, ≥99.0%), D-lactose monohydrate (Sigma-Aldrich, BioUltra, ≥99.5%), D-galactose (Sigma-Aldrich, ≥99%), D-glucose (Sigma-Aldrich, ≥99.5%), silica gel (Sigma-Aldrich, Davisil grade 646), mesoporous SBA-15 (Sigma-Aldrich, <150 μm particle size, 4 and 6 nm pore size), silicon carbide (Sigma-Aldrich, 250-450 mesh particle size), Si-diol (SiliCycle (Québec, Canada), SiliaBond Diol nec, R35030B), Si—C₁ (SiliCycle, SiliaBond C1, R33030B), Si—C₄ (SiliCycle, SiliaBond C4, R32030B), Si—C₁₈ (SiliCycle, SiliaBond C18, R33230B), TiO₂ (TiO₂-P25, Acros Organics (Fairlawn, New Jersey)), sucrose (Sigma-Aldrich, BioXtra, ≥99.5%), acetamide (Sigma-Aldrich, ≥99.0%), Cu/Al₂O₃ (Riogen (Redwood Falls, Minnesota), >98%, 5 wt % Cu loading), 2-propanol (IPA, Fisher chemical, HPLC degree). The above chemicals and materials were used without further purification.

### Synthesis of PHAH by Aldol-Condensation and Hydrogenation:

The aldol-condensed chemical consisting of two HMF (5-hydroxymenthyl furfural) and acetone is denoted as HAH. 1.83 g of NaOH was dissolved in 15 g of Milli-Q water to prepare 3 M of NaOH solution to catalyze aldol-condensation, and 2 mL of 37% HCl and 10 g of Milli-Q water were mixed to prepare 2 M of HCl solution to neutralize the NaOH catalyst after aldol-condensation. 10.2 g of HMF, 3 mL of acetone, and 71.28 mL of Milli-Q water were mixed in a 500 mL round bottom flask with magnetic stifling bar and placed in an oil bath at 35° C. for 5 min. 6.67 mL of 3 M of NaOH solution was added to the HMF and acetone solution, and the flask was capped with a glass lid. After 1 h of aldol-condensation, ˜7.74 mL of 2 M of HCl solution was added to the flask to terminate aldol-condensation by neutralization. A pH strip was used to measure the pH of the aldol-condensed solution. The aldol-condensed solution with the precipitated HAH was vacuum filtered by paper while rinsing with ˜300 mL of Milli-Q water. The washed HAH was dried in a vacuum oven at 50° C., under 500-600 mbar for 2 days.

Partially hydrogenated HAH is denoted as PHAH. 0.250 g of Cu/γ-Al₂O₃ catalyst (5 wt % Cu loading) with a glass-covered magnetic stirring bar was reduced at 300° C. (temperature was ramped from 22 to 300° C. for 2 h) for 1 h under 34 bar (at 22° C.) of H₂ gas in a Parr reactor. HAH feed solution was prepared by dissolving 1.12 g of HAH in an IPA/water cosolvent (IPA/water (mol)=1/1, mixing 19.39 g (24.7 mL) of IPA solvent and 5.75 g (5.8 mL) of MQ water). The HAH feed solution was added by HPLC pump into the 50 mL Parr reactor, containing the reduced Cu/γ-Al₂O₃ catalyst under pressurized H₂ gas (˜30 bar) to avoid catalyst oxidation by air contact. The reactor was purged twice with 50 bar of Ar gas and three times with 30 bar of H₂ gas. The reactor was pressurized to 35 bar of H₂ gas (at 22° C.) and was heated to 120° C. in 35 min (final pressure increased to 45 bar at 120° C.). The reactor was kept at 120° C. for 12 h with 450 rpm of stirring and cooled to room temperature by natural convection. The product solution was separated from the solid catalyst by syringe filter, and the cosolvent was evaporated by rotary evaporation (40° C., 30-100 mbar). The product was characterized by ¹H and ¹³C NMR and was quantified by GC-FID. (12)

### Synthesis of GA-MAL by Imidation:

3.22 g (33 mmol) of maleic anhydride and 4.81 g (33 mmol) of L-glutamic acid were added into 31.5 g (30 mL) of acetic acid solvent in a 200 mL-scale round-bottom flask. The round flask was placed in an oil bath, set at 170° C., with reflux and reacted for 120 min under the stifling (600 rpm). The product solution was cooled to ˜70° C. and the acetic acid solvent was evaporated by a rotary evaporator, set at 70° C., 110 rpm, 50-100 mbar, for 1 h. The concentrated product (orange colored sticky liquid) was placed in a vacuum oven (50° C., 500-600 mbar) for 22 h. The yield of GA-MAL was ˜40 mol % and the concentrated product contained unreacted glutamic acid and maleic acid as a by-product. GA-MAL was purified by chromatography and characterized by ¹³C qNMR and 2D HSQC NMR (data not shown).

### Synthesis of PHAH-GA-MAL by Diels-Alder Reaction:

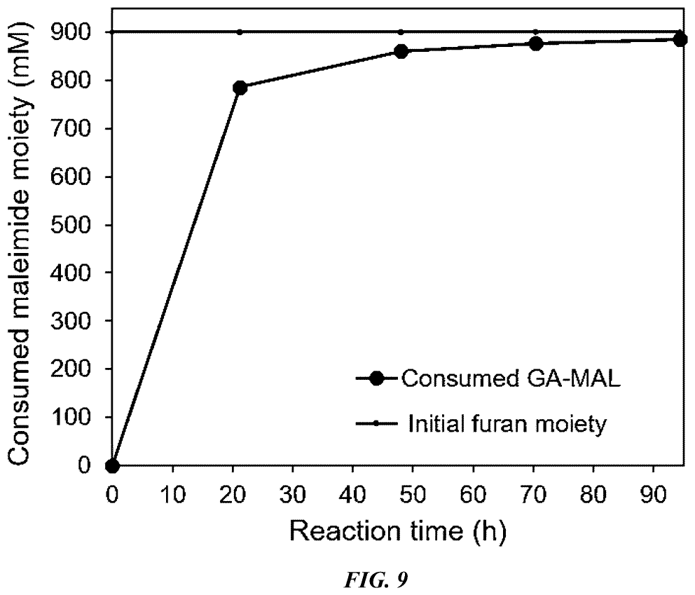

0.36 g (1.3 mmol) of PHAH and 0.58 g (2.6 mmol) of purified GA-MAL were added into 2.52 g (2.8 mL) of THF solvent to prepare a feed solution in a glass vial. The glass vial was capped to prevent the solvent from evaporation, and Diels-Alder reaction was carried out at room temperature (21-23° C.) for 4 days. The conversion of GA-MAL is shown in FIG. 9. THF solvent was evaporated by rotary evaporation (40° C., 30-100 mbar), and PHAH-GA-MAL was characterized by ¹³C qNMR and 2D HSQC NMR (data not shown).

### Synthesis of GA-MAL-H by Hydrogenation:

0.13 g (0.55 mmol) of purified GA-MAL was added into 9.78 g (11 mL) of THF solvent to prepare a feed solution. 0.03 g of Ru/C (5 wt % Ru loading) and 9.86 g of the feed solution were added into a 50 mL-scale Parr reactor. The reactor was purged twice with 45 bar of Ar gas and three times with 30 bar of H₂ gas to remove air in the reactor. The reactor was pressurized to 40 bar of H₂ gas (at 22° C.) and was heated to 30° C. in 20 min (final pressure increased to 42 bar at 30° C.). The reactor was kept at 30° C. for 900 min with 450 rpm of stirring. The product solution was separated from the solid catalyst by syringe filter, and the cosolvent was evaporated by rotary evaporation (40° C., 30-100 mbar). The yield of GA-MAL-H was ˜100 mol % and GA-MAL-H was characterized by ¹³C qNMR and 2D HSQC NMR (data not shown).

### Synthesis of PHAH-GA-MAL-H by Hydrogenation:

0.33 g (0.45 mmol) of the PHAH-GA-MAL was added into 8.00 g (9.0 mL) of THF solvent to prepare a feed solution. 0.06 g of Ru/C (5 wt % Ru loading) and 8.29 g of the feed solution were added into a 50 mL-scale Parr reactor. The reactor was purged twice with 45 bar of Ar gas and three times with 30 bar of H₂ gas to remove air in the reactor. The reactor was pressurized to 40 bar of H₂ gas (at 22° C.) and was heated to 30° C. in 20 min (final pressure increased to 42 bar at 30° C.). The reactor was kept at 30° C. for 900 min with 450 rpm of stirring. The product solution was separated from the solid catalyst by a syringe filter, and the cosolvent was evaporated by rotary evaporation (40° C., 30-100 mbar). The yield of PHAH-GA-MAL-H was ˜100 mol % and PHAH-GA-MAL-H was characterized by ¹³C qNMR and 2D HSQC NMR (data not shown). We note that impurities (e.g. maleic acid) sometimes deactivated the Ru/C catalyst during the first cycle of hydrogenation and showed no conversion of PHAH-GA-MAL. In such cases, the deactivated Ru/C was replaced and a second hydrogenation was repeated to achieve the full conversion of PHAH-GA-MAL.

### Chromatography Separation for the Purification of GA-MAL:

The purification of GA-MAL was performed via column chromatography on a Biotage Isolera One unit with the aid of 120 g of a C₁₈ Biotage Sfär reverse phase column. The separations were performed on 3 g portions of crude material utilizing a water/acetonitrile mobile phase. The mobile phase was swept from 0% to 5% acetonitrile for 7 column volumes, followed by an isocratic region at 5% acetonitrile for 3 column volumes. The column was washed with 100% water and 100% acetonitrile between each run.

### Preparation of Chemical-Coating on Silica and SBA-15 by Pyrolysis:

0.473 g of sucrose was dissolved in MQ water (3.433 g, 6.974 g and 11.45 g of MQ water was used to wet silica, SBA-15 with 4 nm pores, and SBA-15 with 6 nm pores, respectively) to prepare sucrose solutions. Similarly, 0.462 g of acetamide was dissolved in 3.46 g of MQ water to prepare acetamide solution. The sucrose or acetamide solutions were added to 1.799 g of silica or SBA-15 with different pore sizes for wetness incipient impregnation. MQ water was evaporated in a fume hood by stirring the silica slurry for 17-21 h. 1.5 g of the chemical-containing silica was pyrolyzed at 200, 300, and 400° C. for 2 h under Ar gas flow (100 mL/min) to prepare C-coating (11 wt % of carbon) or N-doping (11 wt % of carbon with 6 wt % of nitrogen) of silica supports.

### Immobilization of PHAH-GA-MAL-H on Various Supports:

0.031 g (0.042 mmol) of PHAH-GA-MAL-H was added to 0.163 g of the engineered support in a glass vial. 0.24-0.35 g of THF solvent was added to the glass vial to dissolve PHAH-GA-MAL-H and wet the support. The catalyst slurry was sonicated and vortexed for a few minutes. THF solvent was evaporated by rotary evaporation (40° C., 350-400 mbar) for 70-130 min.

### Reaction Kinetics Analysis for Lactose Hydrolysis:

0.66 g (1.85 mmol) of D-lactose monohydrate was added to 4.10 g of MQ water and heated at 100° C. to dissolve the lactose in water. 4.71 g of the lactose feed solution was added to 0.19 g of immobilized catalyst (or controlled amount of non-immobilized catalysts, such as sulfuric acid, glutamic acid, acetic acid, alanine, tyrosine, taurine, GA-MAL-H, and PHAH-GA-MAL-H) in a glass vial. The glass vial was capped to prevent the solvent from evaporation, and the hydrolysis reaction was carried out at 100° C. in an oil bath (120 and 90° C. were used to measure activation energies) with 600-800 rpm stirring. The glass vial reactor was centrifuged at 2500 rpm for 20 min to separate the immobilized catalyst before sampling. 0.105 g (=0.1 mL) of liquid sample solution was collected at each day (every 18-26 h) for 5 days and diluted in 0.399 g (=0.4 mL) of MQ water for the reaction kinetics analysis, and the diluted sample was filtered by syringe filter (20 μm pore) and analyzed by HPLC. The following equations (Equation 1-4) were used as a kinetic model, and the rate constants for lactose hydrolysis (kₐₚₚ) and glucose degradation (k_{d,app}) were measured by fitting the experimental kinetic data to the kinetic model by MATLAB with 95% confidential intervals.

$$\frac{d [ Lactose ]}{dt} = - {k}_{app} [ Lactose ] \qquad Eq . 1$$

$$\frac{d [ Galactose ]}{dt} = {k}_{app} [ Lactose ] \qquad Eq . 2$$

$$\frac{d [ Glucose ]}{dt} = {k}_{app} [ Lactose ] - {k}_{d , app} [ Degraded Glucose ] \qquad Eq . 3$$

$$\frac{d [ Degraded Glucose ]}{dt} = {k}_{d , app} [ Degraded Glucose ] \qquad Eq . 4$$

$$( [ Degraded Glucose ] = [ Galactose ] - [ Glucose ] )$$

$$( {k}_{app} : apparent rate constant for lactose hydrolysis ,$$

$${k}_{d , app} : apparent rate constant for glucose degradation within 95 \% confidential interval )$$

Each hydrolysis cycle was performed for 5 days (˜110-122 h). After each hydrolysis cycle, the liquid solution was separated from the precipitated catalyst (by centrifuge at 2500 rpm, for 20 min). Then, a refreshed lactose feed solution was prepared by adding 0.66 g of D-lactose monohydrate into 4.10 g of MQ water and heating it at 100° C. to dissolve the lactose in water. 4.71 g of the refreshed feed solution was added to the wet catalyst for the next cycle of the hydrolysis for catalyst stability analysis. Catalyst stability analysis was performed by repeating the hydrolysis cycle for 3-4 times.

### Lactose Hydrolysis in the Presence of Ca(OAc)₂:

The controlled amounts of Ca(OAc)₂ were added to a mixture of 0.974 g (2.70 mmol) of D-lactose monohydrate, 0.080 g (0.54 mmol) of L-glutamic acid, and 6.000 g (6.01 mL) of MQ water (where 0.000 g, 0.0057 g, 0.0172 g, and 0.0251 g of calcium acetate hydrate were added to prepare 0, 5, 16, and 24 mM concentrations of Ca(OAc)₂ in the feed solution, respectively). The glass vial was capped to prevent the solvent from evaporation, and reaction was carried out at 100° C. in an oil bath with 800 rpm stirring. 0.105 g (0.1 mL) of liquid sample solution was collected each day (every 18-26 h) for 5 days and diluted in 0.399 g (0.4 mL) of MQ water for the reaction kinetics analysis. The diluted sample was analyzed by HPLC. Experimentally measured rates at different concentrations of Ca(OAc)₂ were analyzed by the kinetic model (Equations 1-4).

### HPLC Analysis for the Hydrolyzed Lactose Solution:

The chemical concentrations of hydrolyzed lactose solutions were quantified by high performance liquid chromatography (HPLC) analysis. The hydrolyzed solutions with immobilized catalysts were centrifuged (2500 rpm, 20 min) to separate the immobilized catalyst in the aqueous phase solution. 0.105 g (0.1 mL) of the aqueous solution was diluted in 0.399 g (0.4 mL) of Milli-Q water (5 times dilution by volume) to analyze the concentrations of lactose, glucose, galactose, and HMF as one of by-products by glucose degradation. The concentrations of lactose, glucose, galactose, glutamic acid, acetic acid, and GA-MAL-H in aqueous solution were measured by a Water 2695 separation module equipped with an Aminex HPX-87H (Bio-Rad) column and RI detector, while the HMF concentrations were measured with a Waters 2998 PDA detector, set at 320 nm. The temperature of the HPLC column was maintained at 50° C., and the flow rate of the mobile phase (pH 2 water, acidified by sulfuric acid) was 0.6 mL/min.

### NMR Analysis for the Organocatalysts:

¹³C quantitative nuclear magnetic resonance (qNMR), and 2D HSQC NMR spectra were obtained using a Brucker Avance-500 spectrometer. Tetramethylsilane (TMS) (δ: 0 ppm) or deuterated solvents (DMSO-d₆) were used as a reference for chemical shifts. Experimental parameters for 13C qNMR are as followings: Relaxation delay (D1)=20 sec, Acquisition time (AQ)=1 sec, Number of scans (NS)=64, Number of dummy scans (DS)=2, Spectral width (SW)=284 ppm, Middle of spectrum (O1P)=110 ppm, Size of fid (1TD)=71424.

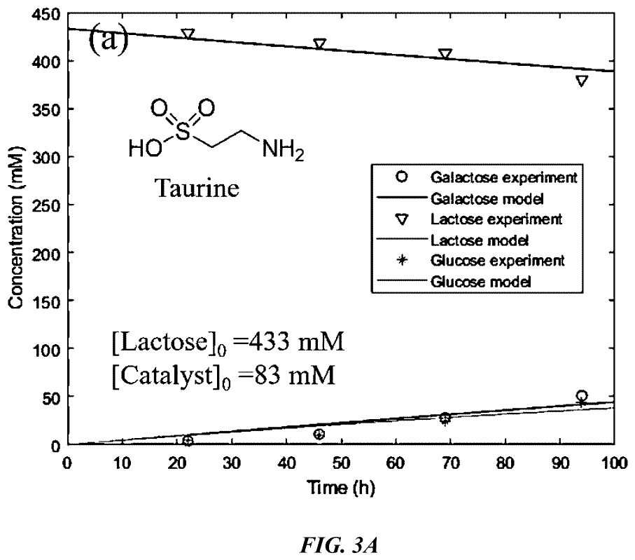

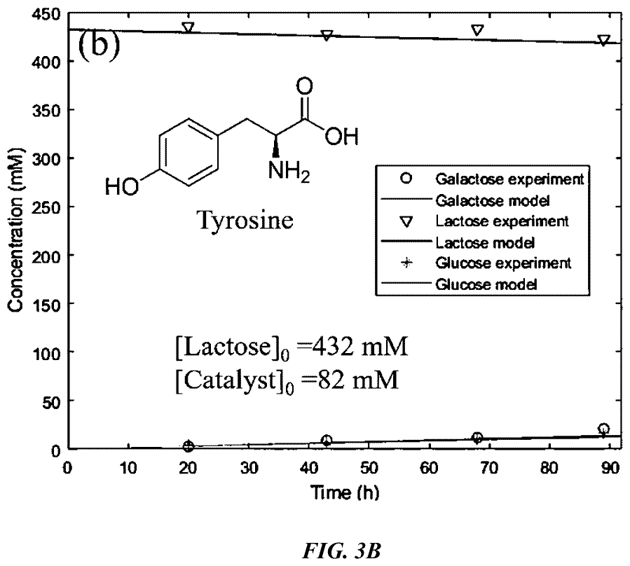

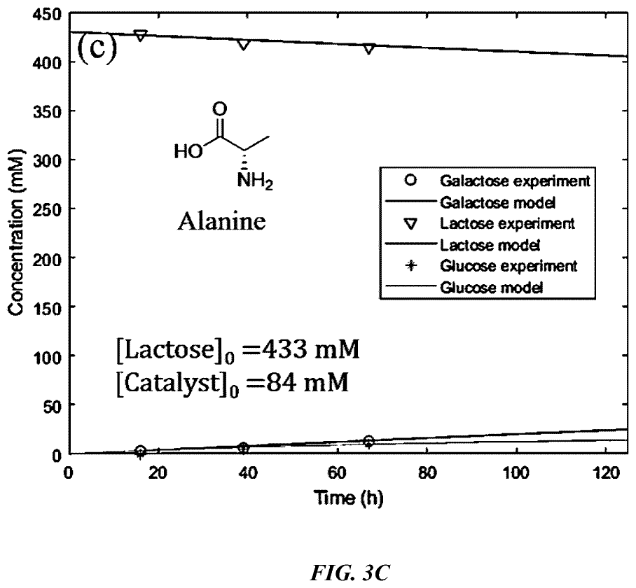

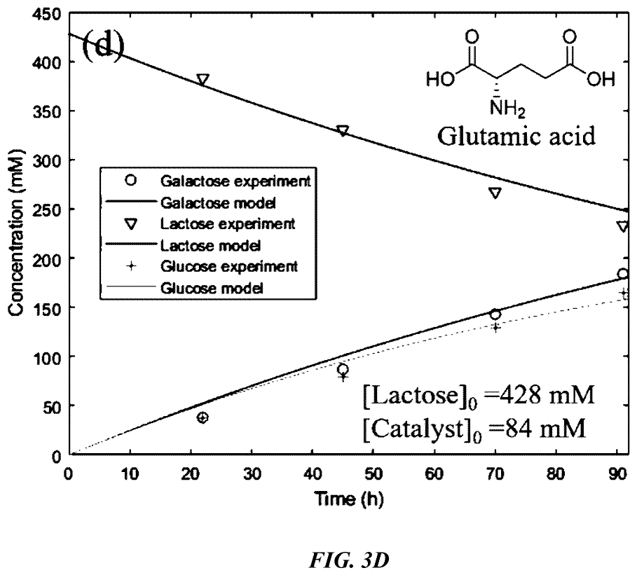

Effect of different amino acids on lactose hydrolysis: Site-directed amino acid substitutions, such as glutamic acid and tyrosine, in enzymes play a role in acid/base active sites for lactose hydrolysis.^{22,23} The catalytic activity for lactose hydrolysis was investigated by reaction kinetics analysis at 100° C. in the presence of amino acids. The concentration of amino acids was held constant (82-84 mM) to measure the catalytic activity (FIG. 3). Glutamic acid showed the highest activity for lactose hydrolysis (kₐₚₚ=0.0060±0.0004 h⁻¹) among the amino acids, including taurine (kₐₚₚ=0.0011±0.0002 h⁻¹), tyrosine (kₐₚₚ=0.0004±0.0001 h⁻¹), and alanine (kₐₚₚ=0.0005±0.0001 h⁻¹). Then, the glutamic acid moiety was appended onto the organocatalysts for the synthesis of biomimetic organocatalysts (FIG. 1A). Glutamic acid has three chemical functional groups: α-carboxylic acid (G₁H), side-chain carboxylic acid (G₂H), and amine (G_{N}) as shown in FIG. 1C. The active site for glutamic acid can be identified by comparing the experimental rates with theoretically predicted rates based on these functionalities.

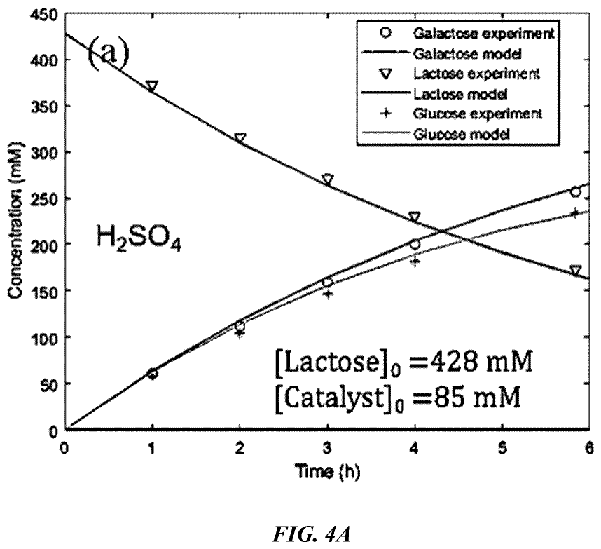

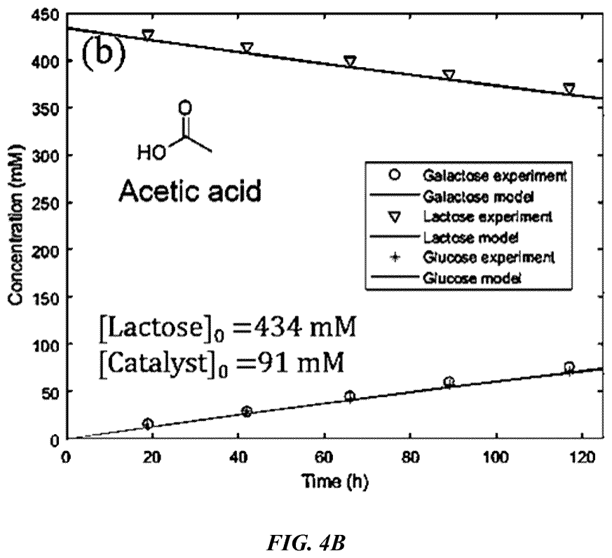

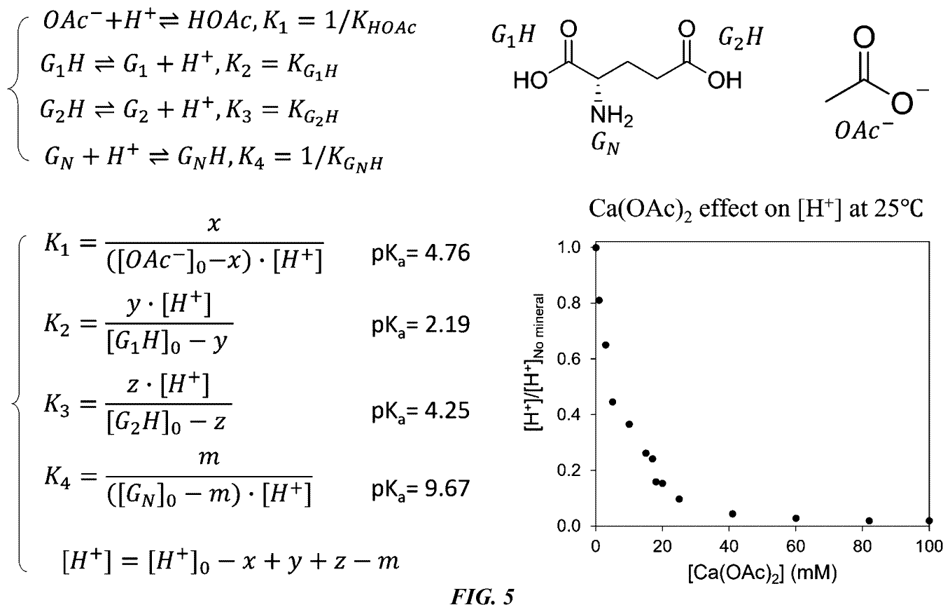

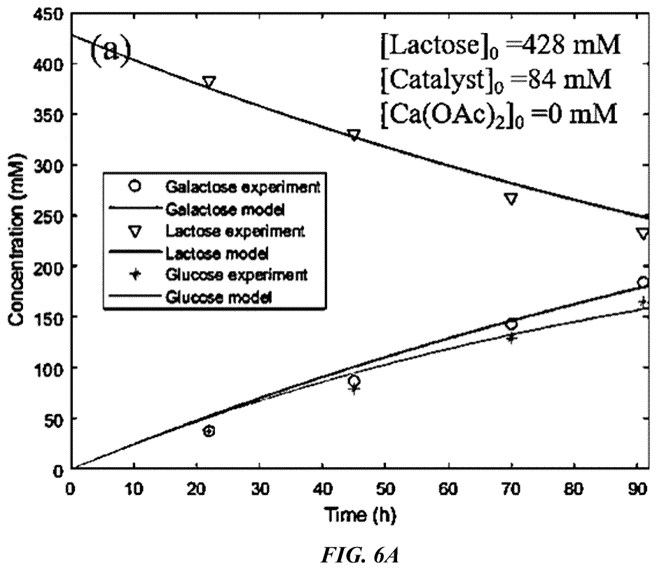

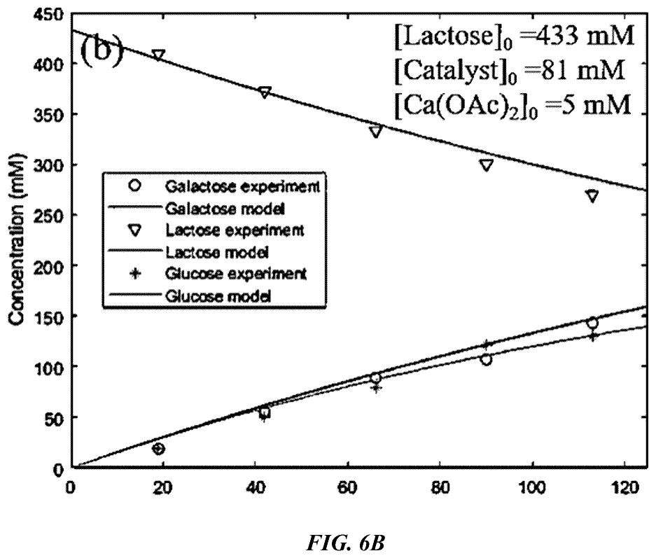

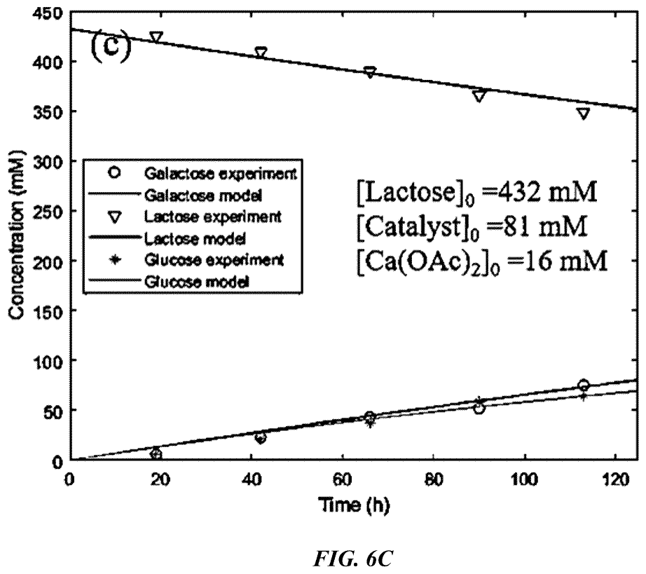

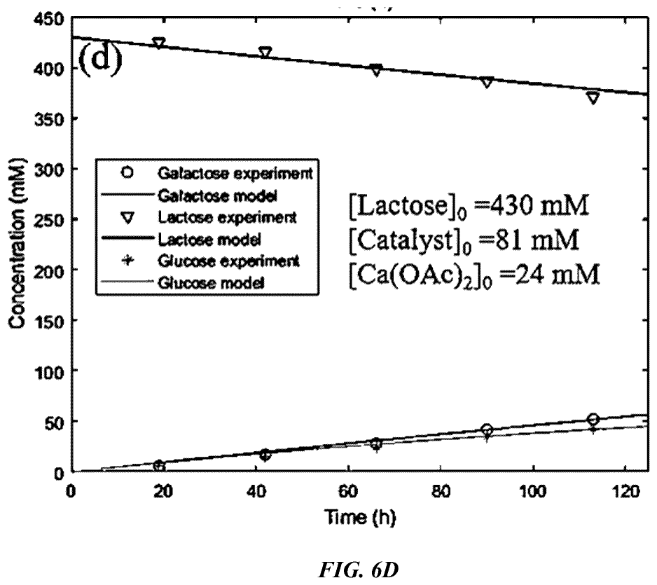

Addition of Ca(OAc)₂ for the investigation of catalytic active sites: The rate constants for H⁺ and non-dissociated carboxylic acid from the acid catalysts were determined by combining results from separate reaction kinetics experiments. The apparent rate constant (kₐₚₚ) for lactose hydrolysis in the presence of H⁺ was experimentally measured by reaction kinetics analysis (FIG. 4A), and the rate constant for H⁺ (k_{H}_{⁺}) was determined to be 0.9500 M⁻¹·h⁻¹ by the following equation, kₐₚₚ=k_{H}_{⁺}·[H⁺]. The addition of Ca(OAc)₂ perturbs the concentration of H⁺ by producing acetic acid (AA), which suppressed the reaction rate for lactose hydrolysis (FIGS. 6A-6D) by shifting thermodynamic equilibrium of protonation (FIG. 5). The concentration of non-dissociated acetic acid ([AA]) was calculated by extrapolating the pKₐ value (pK_{a,AA}=4.87 at 100° C.)²⁴ and the rate constant for the non-dissociated acetic acid (k_{AA}) was determined to be 0.0051 M⁻¹·h⁻¹ by the following equation, kₐₚₚ=k_{H}_{⁺}·[H⁺]+k_{AA}·[AA], and the value of kₐₚₚ was measured by the reaction kinetics analysis in FIG. 4B. Therefore, different amounts of Ca(OAc)₂ can be added to the lactose hydrolysis system to vary the concentration of each active site (e.g., G₁H, G₂H) at constant reaction temperature (at 100° C.).

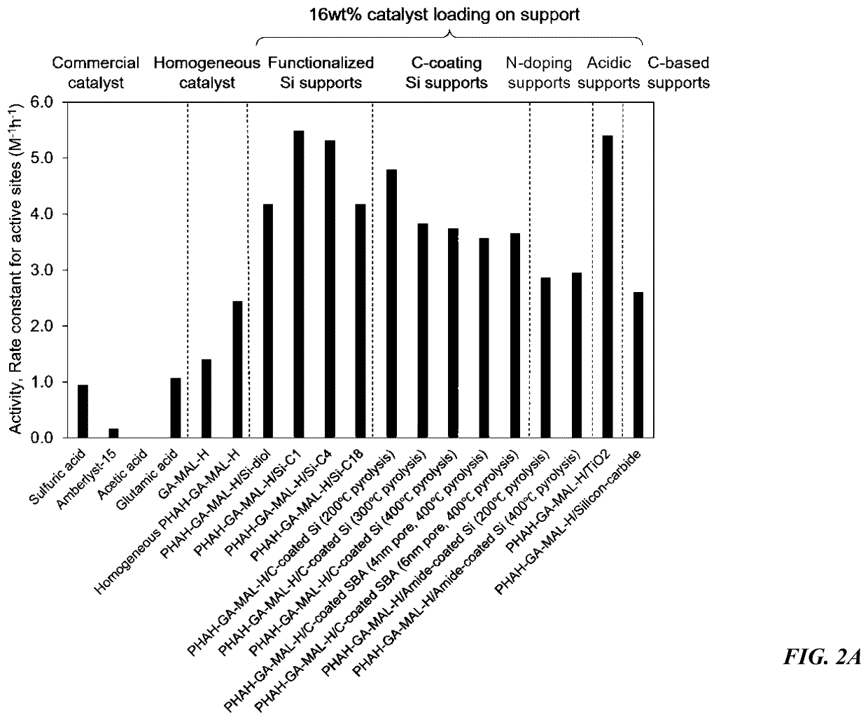

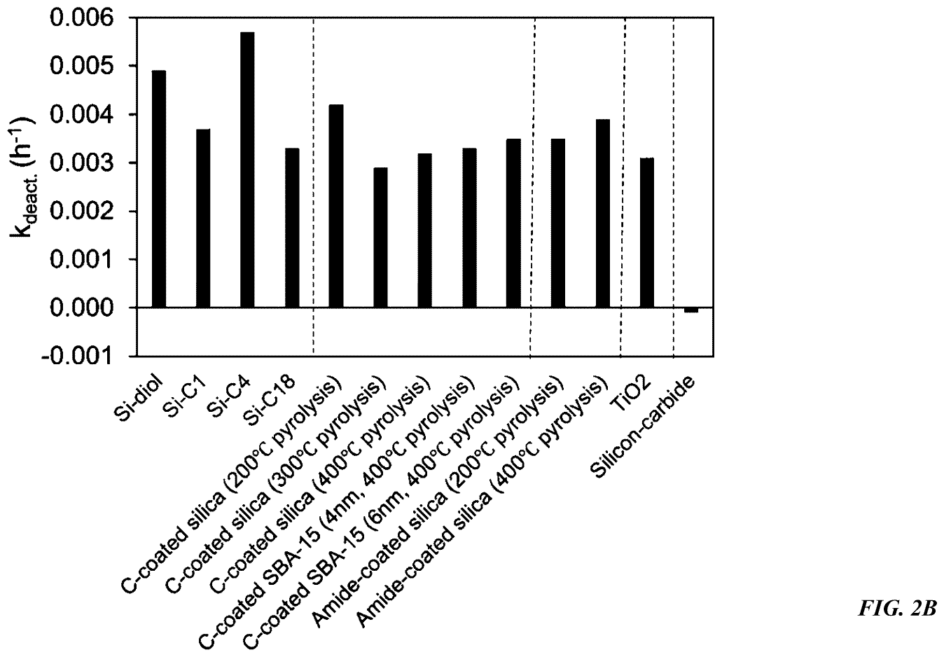

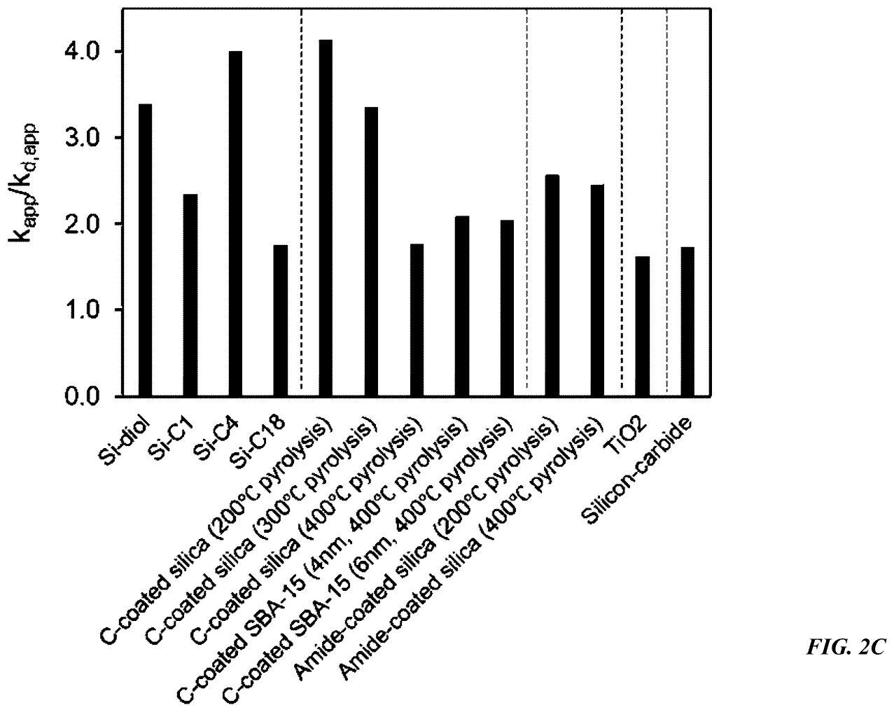

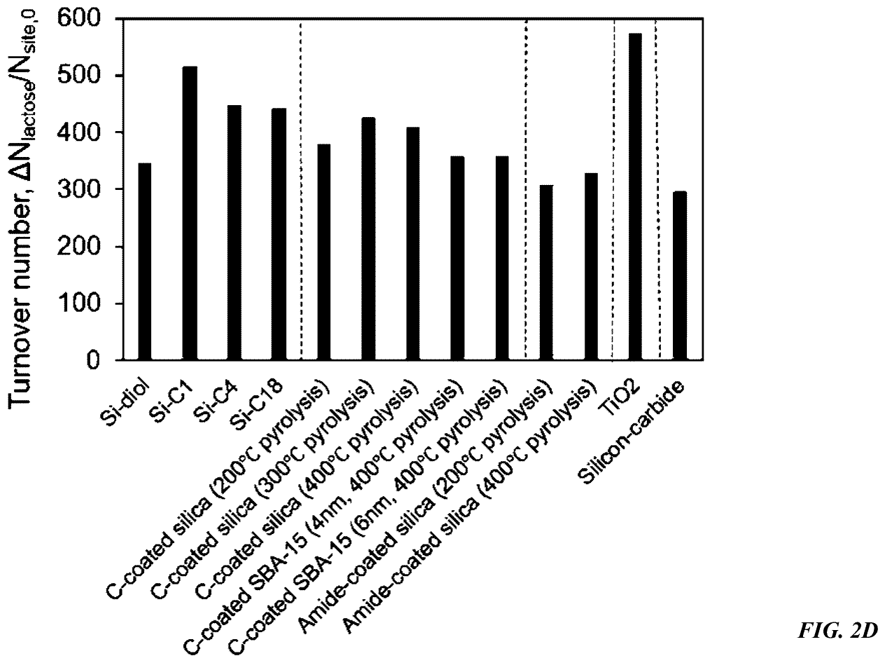

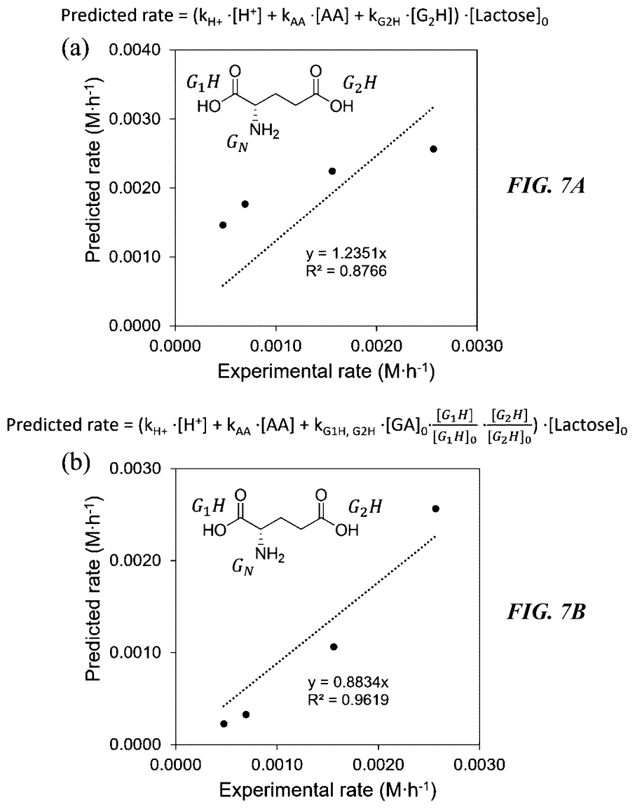

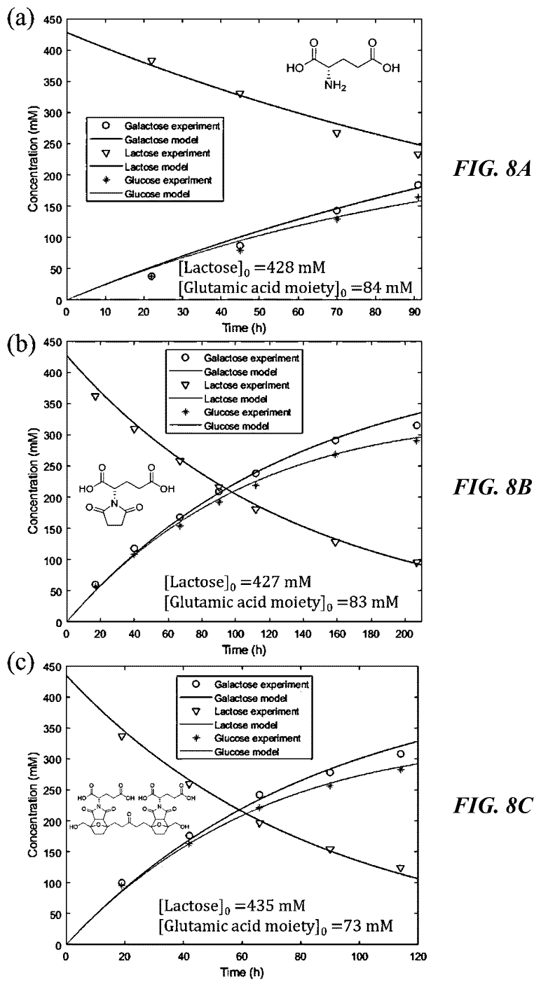

In the presence of different amounts of Ca(OAc)₂, the predicted rates can be calculated by using a linear combination of the rate constants and the concentrations of corresponding active sites (equation in FIG. 1C). The concentrations of [G₁H] and [G₂H] in glutamic acid were calculated by solving the coupled equilibrium equations (in FIG. 5) with the extrapolated pKₐ values (pK_{a,G1H}=2.24, pK_{a,G2H}=4.55, pK_{a,GNH}=9.12 at 100° C.²⁵). The rate constant for G₁H (k_{G}_{₁}_{H}) was determined to be 1.0644 M⁻¹·h⁻¹ by the following equation, kₐₚₚ=k_{H}_{⁺}·[H⁺]+k_{G}_{₁}_{H}·[G₁H], and the value of kₐₚₚ was measured by the reaction kinetics analysis in FIG. 8A. The good fit between the predicted and experimental rates indicates that G₁H is the active site from glutamic acid for lactose hydrolysis, as shown in FIG. 1C (slope=0.9091, R²=0.9454). In contrast, when G₂H or the combination of G₁H and G₂H were assumed to be the active sites, predicted and experimental rates did not match well (FIG. 7). As a result, the rate constants for the active site (G₁H) from the glutamic acid moieties in the presence of biomimetic catalysts were determined by the following equation, k_{G}_{₁}_{H}=(kₐₚₚ−k_{H}_{⁺}·[H⁺])/[G₁H], based on reaction kinetics (e.g. kₐₚₚ, k_{H}_{⁺}) and thermodynamics (e.g. [H⁺], [G₁H]). The k_{G}_{₁}_{H} value for each biomimetic catalyst was then used to calculate the turnover frequency (TOF=k_{G}_{₁}_{H}·[Lactose]₀) for comparison of catalytic activities from the engineered biomimetic catalysts in FIG. 2A. The active site analysis considers two glutamic acid moieties in the di-functional PHAH-GA-MAL-H to count the G₁H sites. This analysis accounts for the contributions from the catalytic activity of H⁺ (pH effect) and the catalytic activity of the active site (G₁H).

Effect of the engineered organocatalysts on lactose hydrolysis: We engineered organocatalysts according to the strategy shown in FIG. 1A, and investigated the effect of the engineered organocatalysts on the catalytic activity for lactose hydrolysis. Glutamic acid was used to produce GA-MAL by imidation of glutamic acid (GA) and maleic anhydride (MAL). The C═C bond in the maleimide moiety of GA-MAL was hydrogenated to produce GA-MAL-H, because hydrogenation increases the stability against hydrothermal degradation of the organocatalyst and prevents side-reactions during lactose hydrolysis at 100° C. Accordingly, GA-MAL-H had 31.9% higher (from 1.06 to 1.40 M⁻¹·h⁻¹) rate constant for the active site (k_{G}_{₁}_{H}) than k_{G}_{₁}_{H} for glutamic acid (FIG. 8A). Glucose degradation was observed as a side-reaction during the lactose hydrolysis. The degree of glucose degradation was quantified by the ratio (k_{d,app}/kₐₚₚ) of apparent rate constant for glucose degradation (k_{d,app}) and lactose hydrolysis (kₐₚₚ), because this ratio represents relative rate of the degradation, based on the rate of lactose hydrolysis. The value of k_{d,app}/kₐₚₚ for GA-MAL-H (k_{d,app}/kₐₚₚ=0.29) was suppressed by 55.2% compared to k_{d,app}/kₐₚₚ for glutamic acid (k_{d,app}/kₐₚₚ=0.45) in FIGS. 8A and 8B.

GA-MAL was further converted to PHAH-GA-MAL by Diels-Alder reaction with a biomass-derived difuran (PHAH) and the conversion of GA-MAL (˜100 mol %) is shown in FIG. 9. During the Diels-Alder reaction, no significant degradation of reactants was observed by NMR and HPLC. The PHAH-GA-MAL complex has a di-functional glutamic acid moiety in its molecular structure. PHAH-GA-MAL was also hydrogenated to saturate the C═C bonds and produce PHAH-GA-MAL-H. The PHAH-GA-MAL-H catalyst displayed 74.2% higher rate constant for the active site (k_{G}_{₁}_{H}=2.45 M⁻¹·h⁻¹) than GA-MAL-H (k_{G}_{₁}_{H}=1.40 M⁻¹·h⁻¹), and the value of k_{d,app}/kₐₚₚ for PHAH-GA-MAL-H (k_{d,app}/kₐₚₚ=0.14) became half of k_{d,app}/kₐₚₚ for GA-MAL-H (=0.29), as shown in FIGS. 8B and 8C. The higher activity and selectivity of GA-MAL-H compared to glutamic acid may result from the imidation that suppresses basicity of the amine group and increases hydrophobicity. R. Breslow, The hydrophobic effect in reaction mechanism studies and in catalysis by artificial enzymes y,z. J. Phys. Org. Chem. 19, 813-822 (2006). The higher activity and selectivity of PHAH-GA-MAL-H than GA-MAL-H implies that the di-functional G₁H site with higher hydrophobicity may lead to the superior catalytic properties because two amino acids substitutions, such as Glu517 and Glu441, in the enzymatic active site contribute to the formation of transition state of lactose for hydrolysis. Rutkiewicz, Bujacz, Wanarska, Wierzbicka-Wos, Cieslinski, Active Site Architecture and Reaction Mechanism Determination of Cold Adapted β-d-galactosidase from Arthrobacter sp. 32cB. Int. J. Mol. Sci. 20, 4301 (2019). Thus, imidation and Diels-Alder reactions of glutamic acid derivatives not only improve the catalytic activity but also suppress the side-reaction during lactose hydrolysis. The molecular structures of the synthesized organocatalysts (GA-MAL, GA-MAL-H, PHAH-GA-MAL, PHAH-GA-MAL-H) were characterized by ¹³C qNMR and 2D HSQC NMR spectra (data not shown). The unassigned chemical shifts in NMR spectrum of PHAH-GA-MAL were derived from PHAH and GA-MAL, formed by retro Diels-Alder reaction during evaporation of THF solvent at 40° C. and the unassigned peaks in the spectrum of PHAH-GA-MAL-H were exo-confirmation of the Diels-Alder adducts.

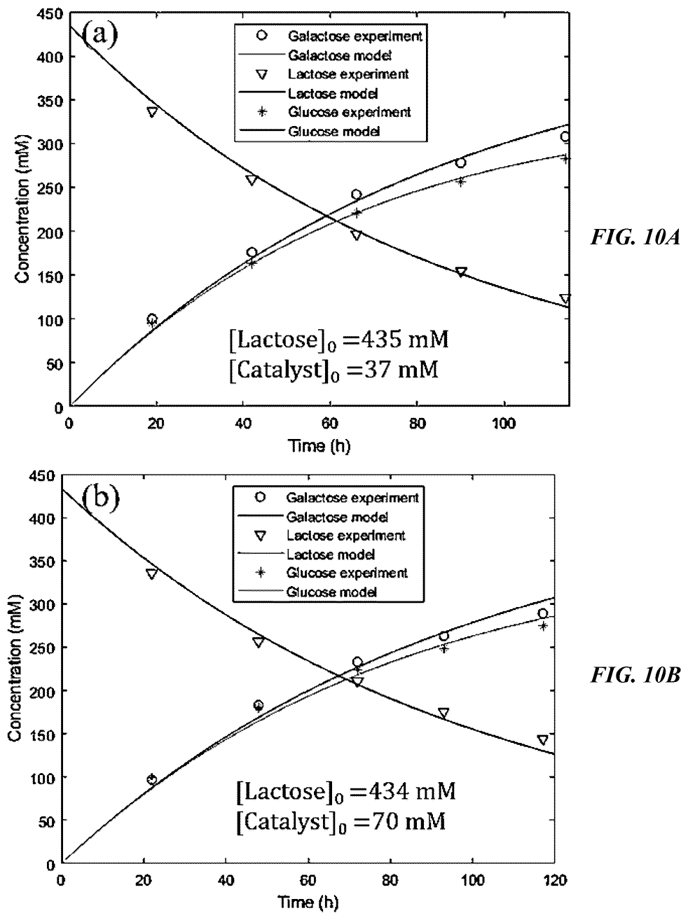

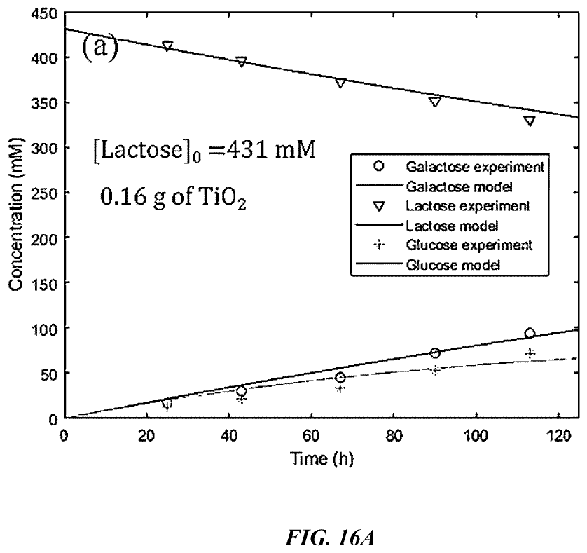

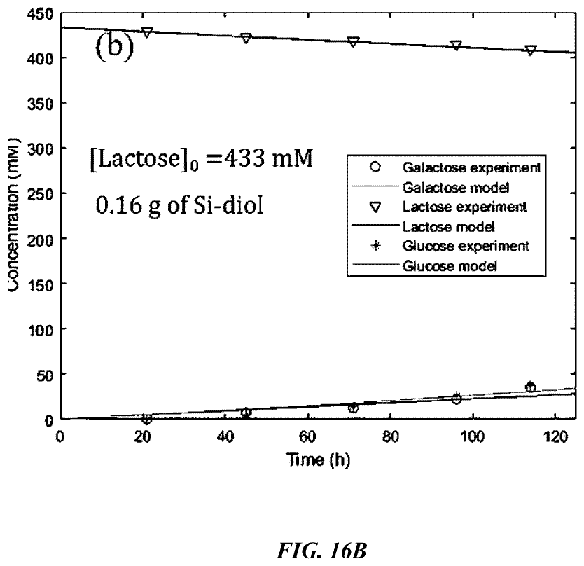

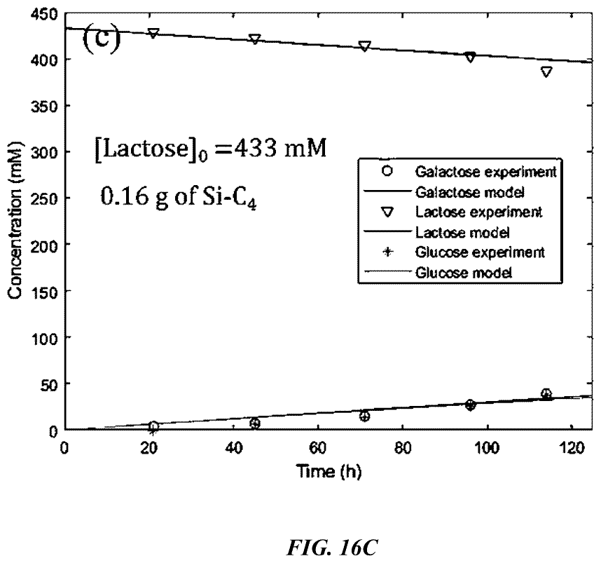

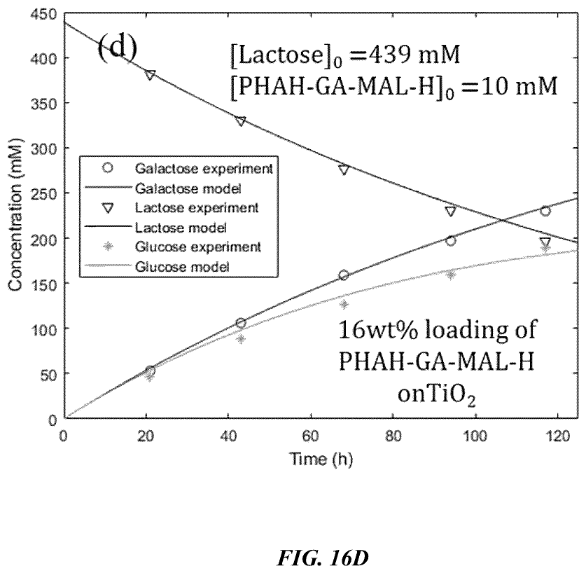

Solubility of the organocatalyst and catalyst deactivation: When the concentration of PHAH-GA-MAL-H increased from 37 to 70 mM, the value of kₐₚₚ remained constant (FIGS. 10A and 10B) because the organocatalyst reached the solubility limit (˜37 mM=27.0 g/L in water at 100° C.) of PHAH-GA-MAL-H in water. Immobilization of the organocatalyst on a solid support material can prevent catalyst precipitation by distributing the organocatalyst on the surface of the support. FIG. 1B shows the complex of the organocatalyst and a well-defined support with functional groups on the surface. Catalytic reaction over the immobilized organocatalyst is analogous to the mechanism of enzymatic reactions. For instance, an organocatalyst complex on a well-defined support with controlled surface properties can act in an analogous fashion as an active site in the channel of an enzyme. Reactants bind on the G₁H sites by the interaction between surface functionality on the support and the organocatalyst. Lower loading of catalyst on a support enhances the catalytic activity (FIGS. 16A and 1D) by forming a more uniform distribution of the complex on the support.

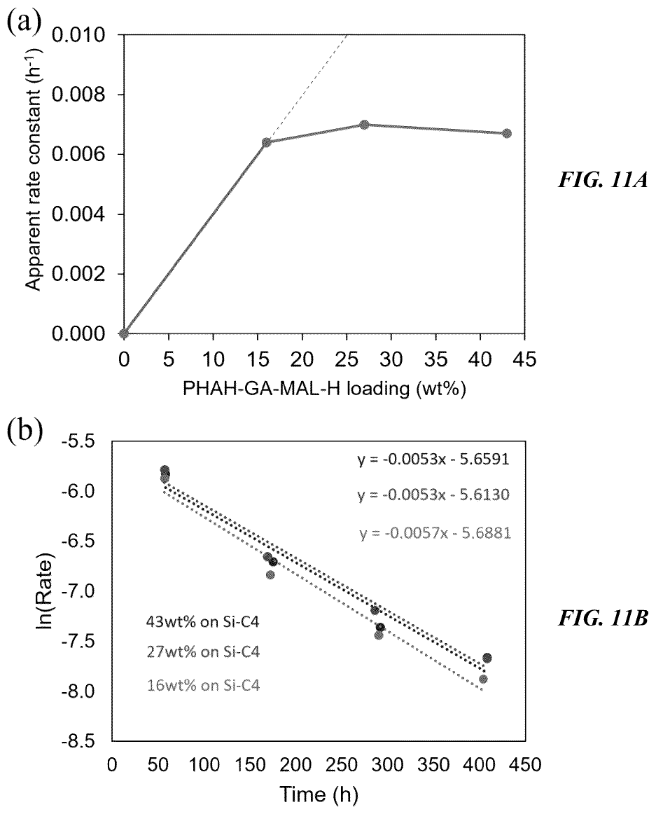

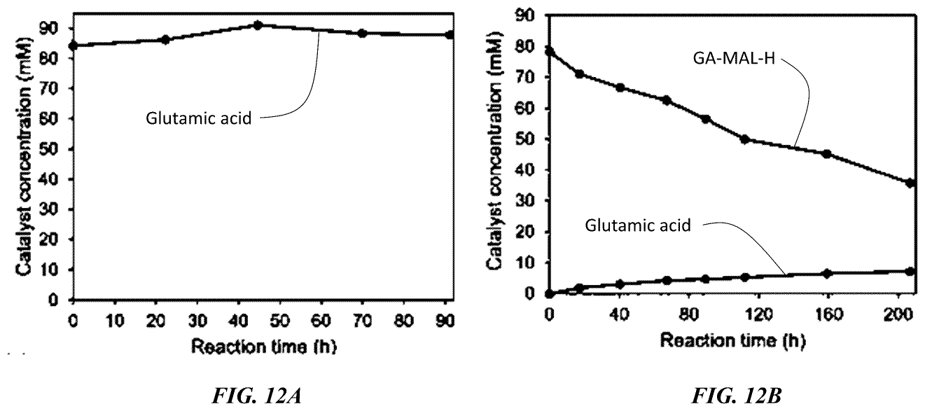

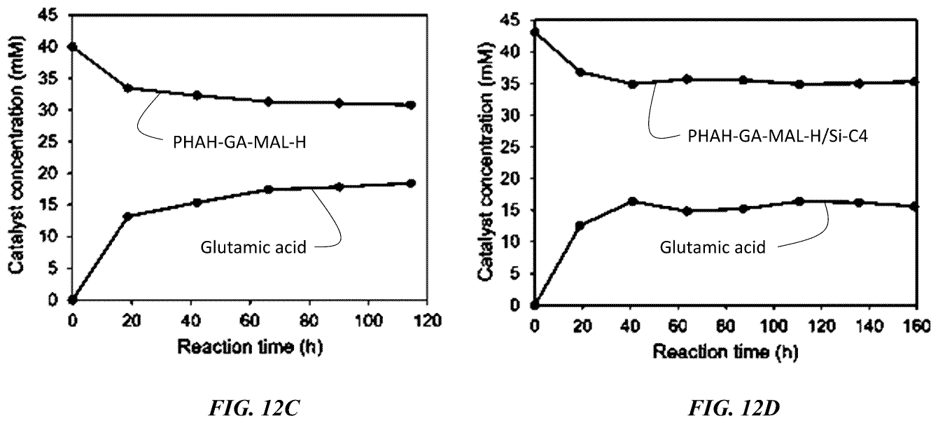

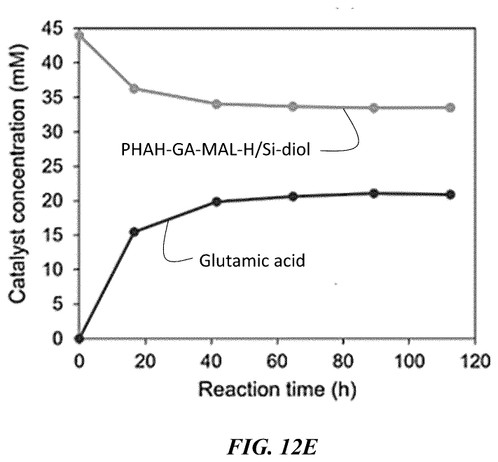

Catalyst deactivation was observed upon the repeated reaction cycles of lactose hydrolysis, but the deactivation is independent of the catalyst loading amount (FIGS. 11A and 11B). The main reason for catalyst deactivation is hydrothermal degradation of the organocatalyst. Glutamic acid was produced during the lactose hydrolysis in the presence of GA-MAL-H and PHAH-GA-MAL-H (FIGS. 12A-12E). The yield of glutamic acid was constant after the immobilization on various functionalized silica supports. This behavior demonstrates that the catalyst immobilization does not prevent catalyst deactivation. Another reason for deactivation is the accumulation of degradation products on the supports as the color of the used immobilized catalyst turned darker during repeated catalytic reaction cycles.

### Analyses of Catalytic Properties in the Presence of Immobilized Organocatalysts

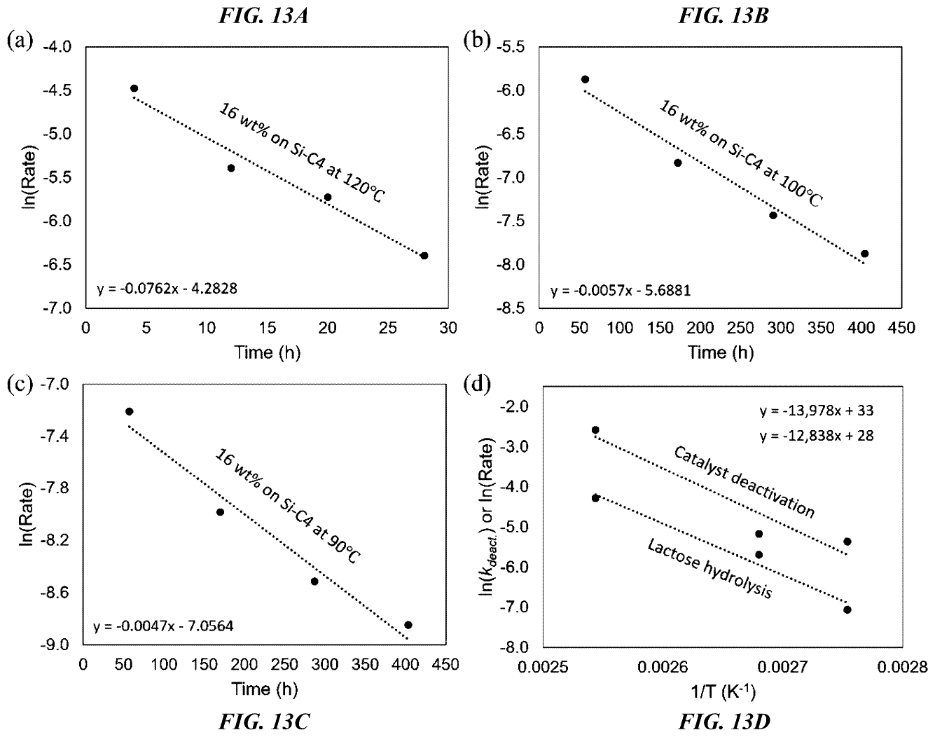

Wetness impregnation has been used to immobilize catalysts on solid supports because catalyst immobilization can improve the stability and activity of the catalysts by preventing hydrothermal decomposition of the catalyst. Various supports were prepared without further characterization and used to immobilize the organocatalyst. The effect of the supports on the catalytic activity (as quantified by the rate constant for the active site) is summarized in FIG. 2A. PHAH-GA-MAL-H (k_{G}_{₁}_{H}=2.45 M⁻¹ h⁻¹) provided 2.3 times higher k_{G}_{₁}_{H} than glutamic acid (k_{G}_{₁}_{H}=1.06 M⁻¹ h⁻¹) which has the highest activity among the commercial chemical catalysts. 16 wt % loading of catalyst immobilization on the methyl-functionalized silica (Si—C₁) support (k_{G}_{₁}_{H}=5.50 M⁻¹ h⁻¹) showed 2.2 times higher TOF than the homogeneous PHAH-GA-MAL-H (k_{G}_{₁}_{H}=2.45 M⁻¹ h⁻¹). The well-defined supports can tailor the catalytic activity by affecting the interaction between lactose and the G₁H active site. We hypothesize that the intermediate hydrophobicity of the support can cause the highest catalytic activity because appropriate hydrophobic and hydrophilic interactions are necessary to mimic enzyme structure, possessing the combination of hydrogen bonds and hydrophobic channel from aromatic residues that constructs the enzymatic active site. Our experiments show that the silica support functionalized with —C₁ (k_{G}_{₁}_{H}=5.50 M⁻¹ h⁻¹) and —C₄ groups (k_{G}_{₁}_{H}=5.32 M⁻¹ h⁻¹) had ˜30% higher TOF than -diol (k_{G}_{₁}_{H}=4.18 M⁻¹ h⁻¹) and —C₁₈ functionalized silica (k_{G}_{₁}_{H}=4.18 M⁻¹ h⁻¹). These results demonstrate that intermediate hydrophobicity (—C₁ and —C₄) of the functionalized silica enables the best catalytic activity. The density of —OH groups on silica and alumina supports can catalyze glucan hydrolysis by the formation of hydrogen bond between the Brønsted acidic —OH sites and the glycosidic oxygen. Pyrolysis of diverse chemicals, such as sucrose and acetamide, over silica can provide carbon (C) coating or nitrogen-atom (N) doping on the surface of the support. Moreover, pyrolysis temperature affected the surface functionality of the chemical-coating supports and tailored the catalytic activity by tailoring the amounts of —OH groups. Pyrolysis temperature at 200° C. resulted in intermediate hydrophobicity of the C-coated silica surface and yielded higher k_{G}_{₁}_{H} (k_{G}_{₁}_{H}=4.80 M⁻¹ h⁻¹) than the C-coating support treated by higher pyrolysis temperature (k_{G}_{₁}_{H}=3.83 and 3.74 M⁻¹ h⁻¹ for 300 and 400° C. pyrolysis). The controlled porous structure of silica (SBA-15) with different pore sizes (4 and 6 nm) did not show a difference in catalytic activity (k_{G}_{₁}_{H}=3.74, 3.57 and 3.66 M⁻¹ h⁻¹ for random pore silica, SBA-15 with 4 nm pore, and 6 nm pore, respectively). N-doping on the support displayed 61.6% lower k_{G}_{₁}_{H} (k_{G}_{₁}_{H}=2.87-2.95 M⁻¹ h⁻¹) than C-coated supports (k_{G}_{₁}_{H}=3.57-4.80 M⁻¹ h⁻¹) suggesting that N-doping sites may serve as basic sites. An acidic support (TiO₂)²⁹ had a value of k_{G}_{₁}_{H} (k_{G}_{₁}_{H}=5.41 M⁻¹ h⁻¹) as high as Si—C₁ support likely due to the acidic properties of the TiO₂ support where 32.3% of the apparent rate constants for hydrolysis (kₐₚₚ) over PHAH-GA-MAL-H supported on TiO₂ resulted from the bare TiO₂ support (FIGS. 13A-13D). Carbon-based supports (e.g., silicon-carbide) had lower k_{G}_{₁}_{H} (k_{G}_{₁}_{H}=2.60 M⁻¹ h⁻¹) than the functionalized silica supports (k_{G}_{₁}_{H}=4.18-5.50 M⁻¹ h⁻¹).

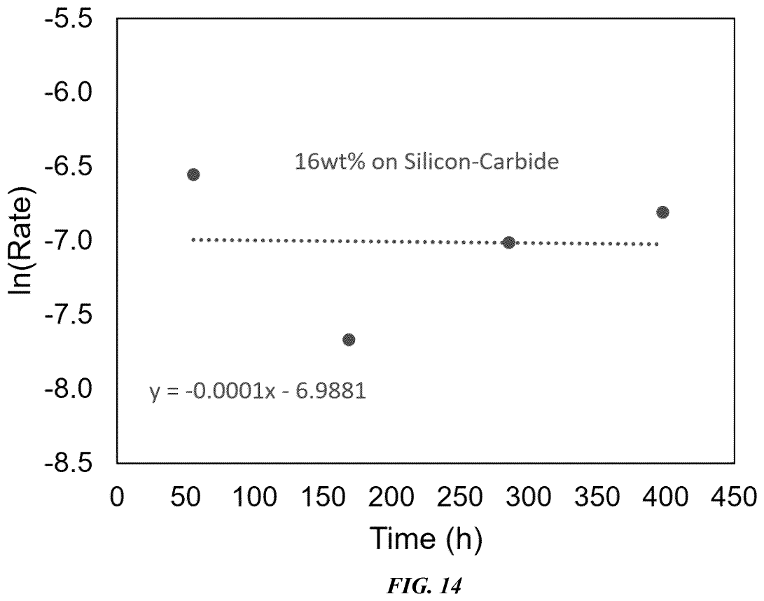

FIGS. 2B, 2C and 2D summarize the catalytic stability, selectivity, and initial turnover numbers for the biomimetic organocatalysts. The silicon-carbide support was the only support with no catalyst deactivation (FIGS. 2B and 14). All other supports represented similar degree of catalyst deactivation (k_{deact.}=0.0030-0.0066 h⁻¹). Catalyst selectivity was assessed by the ratio (kₐₚₚ/k_{d,app}) between the rate of the target reaction (lactose hydrolysis, kₐₚₚ) and the rate of the undesired reaction (glucose degradation, k_{d,app}), measured by kinetic analysis of the first cycle of lactose hydrolysis (FIG. 2C). Surfaces with intermediate hydrophobicity, such as Si—C₄ and C-coated Si (treated by 200° C. pyrolysis) showed the highest selectivity (kₐₚₚ/k_{d,app}˜4.00). Total turnover numbers

$$( T O N = \frac{Δ {N}_{lactose}}{{N}_{site , 0 )}} )$$

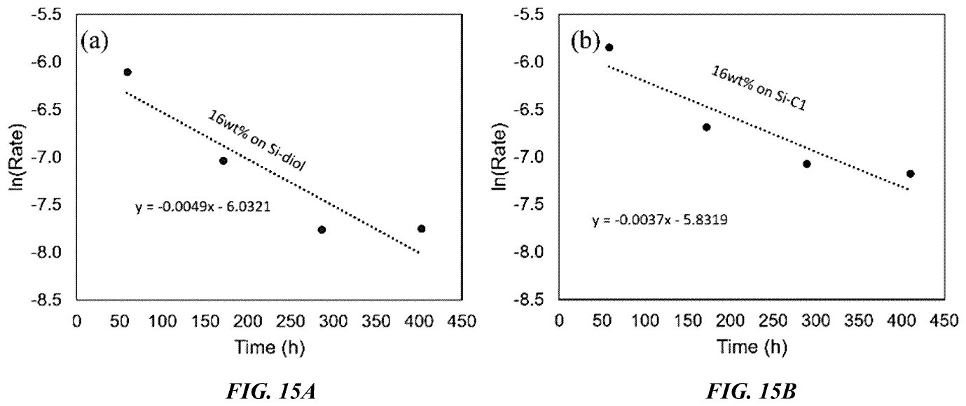

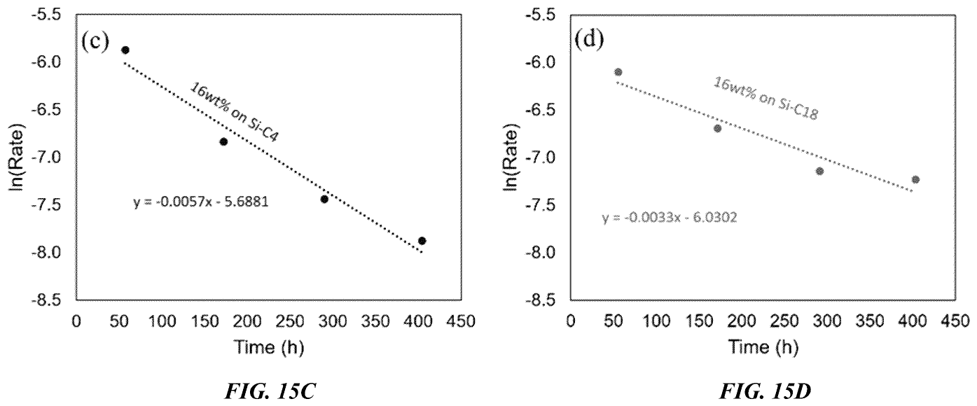

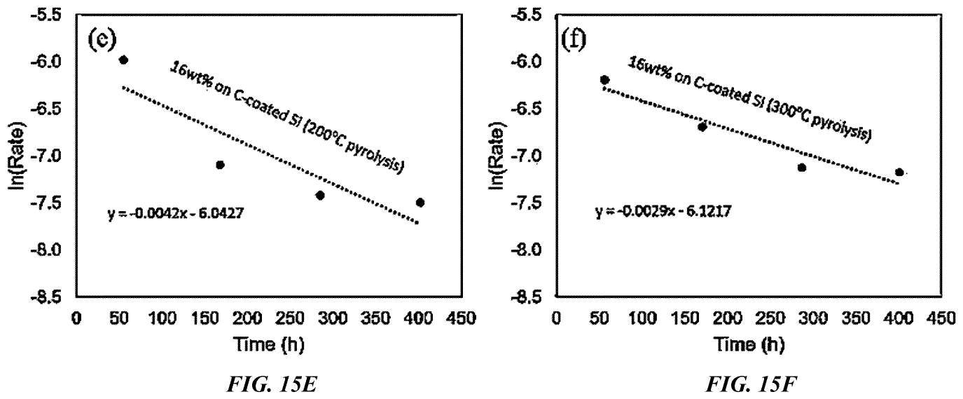

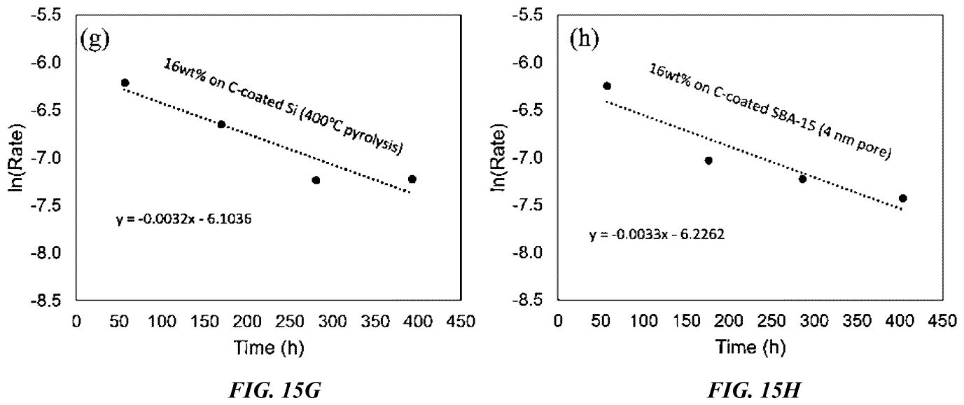

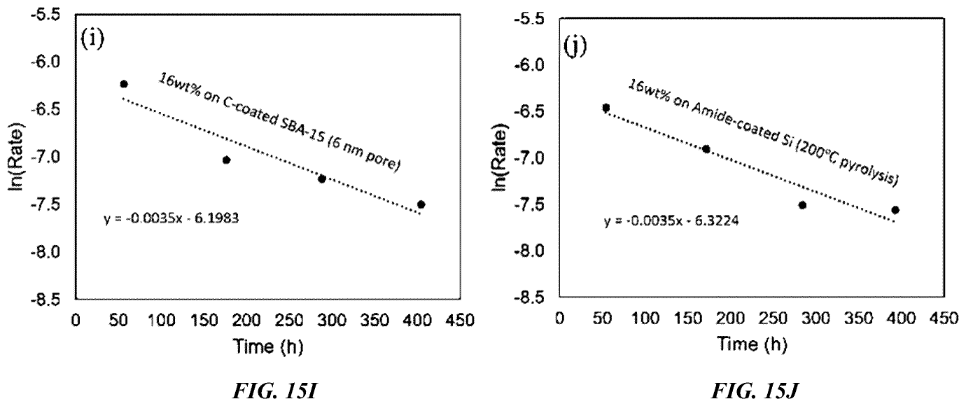

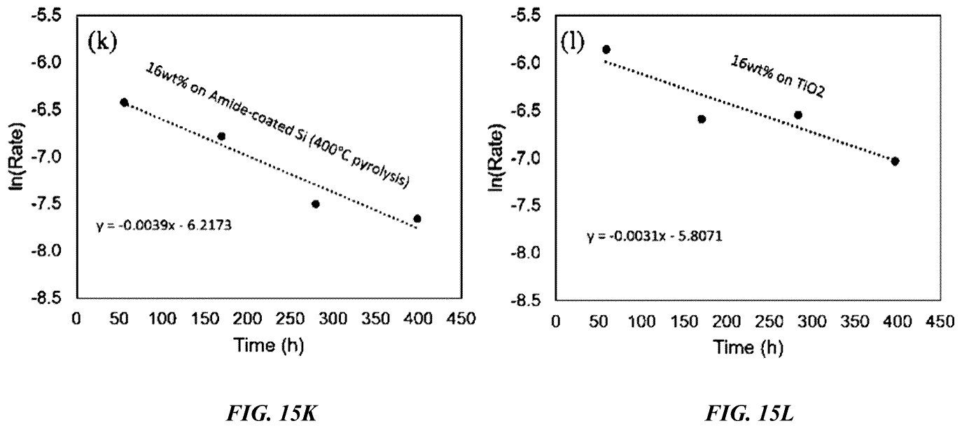

of the deactivating catalysts over ˜400 h on stream were calculated by Equations 6 (rearranging Equation 5, with t=400 h). k_{deact.} and initial rates for lactose hydrolysis were determined from the slope and the y-intercept (=ln(Rate₀)) of catalyst stability plots (FIGS. 15A-15L), and the values of Rate₀ were used to calculate k_{G}_{₁}_{H} and TOF for the active sites. Total turnover numbers

$$( TON = \frac{Δ {N}_{lactose}}{{N}_{site , 0 )}} )$$

or the non-deactivating catalysts were calculated by Equation 7.

$$Δ {N}_{lactose} = {\int }_{0}^{t} Rate dt = T O F \cdot {N}_{site , 0} {\int }_{0}^{t} {e}^{- {k}_{deact .} t} dt \qquad Eq . 5$$

$$T O N = \frac{Δ {N}_{lactose}}{{N}_{site , 0}} = T O F \cdot {\int }_{0}^{t} {e}^{- {k}_{deact .} t} d t = T O F \cdot ( \frac{1 - {e}^{- {k}_{deact .} t}}{{k}_{deact .}} ) \qquad Eq . 6$$

$$T O N = \frac{Δ {N}_{lactose}}{{N}_{site , 0 )}} = T O F from averaged rate ( {h}^{- 1} ) \cdot Operation time ( h ) \qquad Eq . 7$$

In FIG. 2D, total turnover numbers for the immobilized catalysts on amide-coated silicas (TON=310-330) and silicon-carbide supports (TON=295) were lower than functionalized silica (TON=350-515) and C-coated silica (TON=360-425). Similar to the trends in activity and selectivity in FIGS. 2A and 2C, it appears that supports with intermediate hydrophobicity, such as Si—C₁ and C-coated silica at 300° C. pyrolysis, yielded the highest total turnover numbers. Bare supports without the immobilized catalyst had negligible catalytic activities (kₐₚₚ=0.0005-0.0008 h⁻¹) for lactose hydrolysis, except the acidic support (TiO₂) (FIGS. 16A-16D), and we note that the turnover numbers for PHAH-GA-MAL-H on TiO₂ included the effect of acidic support.

### Effect of Reaction Temperature on Catalytic Properties

Decreasing the reaction temperature to 80° C. significantly decreased the rate of lactose hydrolysis, whereas increasing the temperature did not prevent catalyst deactivation. Moreover, increasing temperature does not improve the relative rate of lactose hydrolysis versus catalyst deactivation because the activation energy for lactose hydrolysis (107 kJ/mol) and catalyst deactivation (116 kJ/mol) are similar (FIGS. 13A-13D). The used PHAH-GA-MAL-H, immobilized on Si—C₄ support (43 wt % loading) was recollected by extraction in ethyl acetate (EtOAc) solvent after the 5th cycle of lactose hydrolysis and characterized by 2D HSQC NMR (data not shown). The recollection yield of the used PHAH-GA-MAL-H from the support was ≤5.03 wt %, indicating that the organocatalyst degraded and accumulated on the support during the lactose hydrolysis.

What is claimed is:

1. An organocatalyst comprising a structure: 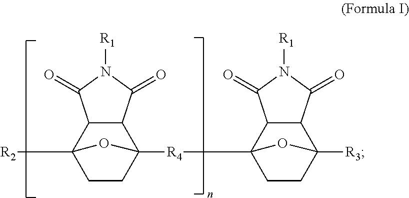
wherein each R₁, independent of every other R₁, is selected from the group consisting of hydrogen, halogen, optionally substituted alkyl, optionally substituted alkenyl, optionally substituted alkynyl, optionally substituted cycloalkyl, optionally substituted cycloalkenyl, hydroxyl, carboxyl, dicarboxyl, optionally substituted alkyloxy, optionally substituted alkenyloxy, optionally substituted alkynyloxy, optionally substituted cycloalkyloxy, optionally substituted cycloalkenyloxy, thiol, optionally substituted alkylthio, optionally substituted alkenylthio, optionally substituted alkynylthio, optionally substituted alkylsulfinyl, optionally substituted alkylsulfonyl, optionally substituted alkylsulfonyloxy, optionally substituted cycloalkylthio, optionally substituted cycloalkylsulfinyl, optionally substituted cycloalkylsulfonyl, optionally substituted cycloalkylsulfonyloxy, optionally substituted cycloalkenylthio, optionally substituted cycloalkenylsulfinyl, optionally substituted cycloalkenylsulfonyl, optionally substituted cycloalkenylsulfonyloxy, optionally substituted amino, acyl, optionally substituted alkyloxycarbonyl, optionally substituted alkenyloxycarbonyl, optionally substituted alkynyloxycarbonyl, optionally substituted aryloxycarbonyl, optionally substituted carbamoyl, optionally substituted sulfamoyl, cyano, nitro, optionally substituted aryl, optionally substituted aryloxy, optionally substituted arylthio, optionally substituted arylsulfinyl, optionally substituted arylsulfonyl, optionally substituted arylsulfonyloxy, optionally substituted heteroaryl, optionally substituted heteroaryloxy, optionally substituted heteroarylthio, optionally substituted heteroarylsulfinyl, optionally substituted heteroarylsulfonyl, optionally substituted heteroarylsulfonyloxy, and an optionally substituted non-aromatic heterocyclic group;
R₂ and R₃ are independently selected from the group consisting of hydrogen, hydroxy, halo, carboxyl, amine, alkyl, alkoxy, hydroxyalkyl, acyl, aryl, heteroalkyl, heteroalkoxy, heteroacyl, and heteroaryl;
R₄ if present is a linker; and
“n” is an integer equal to or greater than 0.

2. The organocatalyst of claim 1, wherein R₁ is a chemical functionality that catalyzes a chemical reaction.

3. The organocatalyst of claim 1, wherein R₁ is selected from the group consisting of carboxyl, dicarboxyl, optionally substituted amino, acyl, optionally substituted alkyloxycarbonyl, optionally substituted aryl, optionally substituted aryloxy, optionally substituted heteroaryl, and optionally substituted non-aromatic heterocyclic group.

4. The organocatalyst of claim 1, wherein each R₁ and the maleimide ring to which it is attached is independently an imidation reaction product of an amine-containing compound and maleic anhydride.

5. The organocatalyst of claim 4, wherein the amine-containing compound is an amino acid.

6. The organocatalyst of claim 4, wherein the amine-containing compound is selected from the group consisting of glutamic acid, tyrosine, taurine, and alanine.

7. The organocatalyst of claim 4, wherein the amine-containing compound is glutamic acid.

8. The organocatalyst of claim 1, wherein each R₁ is independently a Gabriel synthesis reaction product of a protected maleimide and a reactant comprising a primary alkyl halide moiety.

9. The organocatalyst of claim 1, wherein R₂ and R₃ are each hydroxy-C₁-C₆-alkyl.

10. The organocatalyst of claim 1, wherein R₄ is selected from the group consisting of alkyl, alkoxy, carboxy, acyl, and amino.

11. The organocatalyst of claim 1, wherein R₄ is —(CH₂)₁₋₆—CO—(CH₂)₁₋₆—.

12. The organocatalyst of claim 1, having a structure:

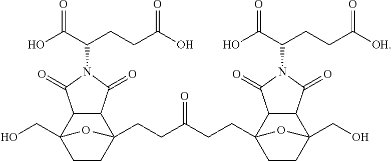

13. The organocatalyst of claim 1, further comprising, in combination, a support, wherein the organocatalyst is immobilized on the support.

14. A method of making an organocatalyst, the method comprising:
(a) reacting an N-substituted maleimide with a furan-containing compound via a Diels-Alder reaction to yield an intermediate; and then
(b) hydrogenating the intermediate of step (a), to yield the organocatalyst.

15. The method of claim 14, further comprising, prior to step (a), forming the N-substituted maleimide by reacting an amine-containing compound with maleic anhydride by imidation to yield the N-substituted maleimide.

16. The method of claim 15, wherein the amine-containing compound is an amino acid.

17. The method of claim 16, wherein the amino acid is selected from the group consisting of glutamic acid, tyrosine, taurine, and alanine.

18. The method of claim 16, wherein the amino acid is glutamic acid.

19. The method of claim 14, wherein the furan-containing compound is a furan having one furan ring or a saturated analog thereof.

20. The method of claim 14, wherein the furan-containing compound is a compound having two or more furan rings or a saturated analog thereof.

21. The method of claim 14, wherein the furan-containing compound is synthesized via an aldol condensation and hydrogenation of 5-hydroxymethyl furfural with acetone.

22. The method of claim 21, wherein an —OH group of the furan-containing compound is oxidized to an aldehyde moiety, and then reacting the aldehyde moiety with acetone and 5-hydroxymethyl furfural in an aldol condensation reaction.

23. The method of claim 14, wherein the furan-containing compound is synthesized by sequential aldol condensation of 2,5-diformylfuran with acetone.

24. The method of claim 14, wherein the furan-containing compound is a furan-containing oligomer or furan-containing polymer synthesized from furan-containing monomer.

25. The method of claim 14, further comprising immobilizing the organocatalyst of step (ii) on a support.

26. The method of claim 14, further comprising, prior to step (a), forming the N-substituted maleimide by reacting a protected maleimide and a primary alkyl halide moiety.
